(19) United States
(12) Patent Application Publication
(10) Pub. No.: US 2022/0370718 A1
(43) Pub. Date: Nov. 24, 2022

(54) DEVICES, SYSTEMS AND METHODS FOR MEDICAMENT DELIVERY

(71) Applicant: MACH32 INC., Edmonton (CA)

(72) Inventors: Marc CURIAL, Edmonton (CA); Christopher TERRIFF, Edmonton (CA); Will COMEAU, Edmonton (CA); Ryley MCCONKEY, Waterloo (CA)

(21) Appl. No.: 17/773,567

(22) PCT Filed: Nov. 4, 2020
(86) PCT No.: PCT/CA2020/051495

§ 371 Date: Apr. 29, 2022

Related U.S. Application Data
(60) Provisional application No. 62/930,276, filed on Nov. 4, 2019.

(51) Int. Cl. A61M 5/24; A61M 5/31; A61M 5/315; A61B 17/34; A61M 5/145; A61M 1/00; A61M 5/20; A61M 5/168

(52) U.S. Cl. CPC A61M 5/2429; A61M 5/3148; A61M 5/3146; A61M 5/31511; A61B 17/3472; A61M 5/1452; A61M 1/774; A61M 5/2033; A61M 5/16813; A61M 5/3158

(57) ABSTRACT

The present disclosure is generally directed to a delivery device housed within an outer cartridge and useful for delivering a fluid to the IO space of a subject at a relatively constant volumetric flow rate over an extended period of time. The delivery device may be fluidly coupled to an existing IO access device or IV line positioned in the IO space of the subject or may be coupled to an IO access device disposed within the outer cartridge.

### CROSS-REFERENCE TO RELATED APPLICATION

This application claims the benefit of U.S. Provisional Patent Application Ser. No. 62/930,276 filed on Nov. 4, 2019. The contents of the aforementioned application are incorporated by reference herein.

### FIELD

The present disclosure generally relates to a medical device, and more particularly to a delivery device for automatically injecting a fluid into a target site of a subject, such as, but not limited to, the intraosseous space of a bone, after activation of the delivery device.

### BACKGROUND

Every year many subjects are treated for life-threatening emergencies. Such emergencies can include shock, trauma, cardiac arrest, drug overdoses, diabetic ketoacidosis, arrhythmias, burns, and status epilepticus just to name a few. An essential element for treating many of these life threatening emergencies is rapid establishment of an IV line in order to administer drugs and fluids directly into a subject's vascular system. Whether in an ambulance by paramedics, in an emergency room by emergency specialists or on a battlefield by an Army medic, the goal is the same—quickly start an IV in order to administer lifesaving drugs and fluids. To a large degree, the ability to successfully treat most critical emergencies is dependent on the skill and luck of an operator in accomplishing vascular access. Doctors, nurses and paramedics can experience great difficulty in establishing IV access in many subjects due to a variety of causes, such as subjects with chronic disease or subjects that may not have available IV sites due to anatomical scarcity of peripheral veins, obesity, extreme dehydration or previous IV drug use. A further complicating factor in achieving IV access occurs “in the field” e.g. at the scene of an accident or military combat, or during ambulance transport where it is difficult to see the target and excessive motion makes accessing the venous system difficult.

The intraosseous (IO) space provides a direct conduit to a subject's vascular system and provides an attractive alternate route to administer IV drugs and fluids. Drugs administered intraosseously enter a subject's blood circulation system as rapidly as they do when given intravenously. In essence, bone marrow may function as a large non-collapsible vein.

Known devices capable of accessing the IO space and/or administering drugs intraosseously include, for example, the devices described in:
U.S. Pat. Nos. 3,750,667 and 3,893,445 which disclose access devices that include a cutting blade which bores a hole into the bone as the device is being rotated and threading the device through the hole in a single step;
U.S. Pat. No. 4,772,261 which discloses an access device capable of boring a small hole into the bone, and screwing the device into the bone through the hole;
U.S. Pat. No. 5,122,114 which discloses an access device that can be installed using either the pre-boring method or the bore-as-threaded method described above;
U.S. Pat. Nos. 5,176,643, 5,451,210 and 7,811,260 which disclose devices capable of accessing the IO space using a needle drive assembly and administering medication into the IO space within seconds; and
U.S. Pat. Nos. 5,312,364, 5,817,052 and 6,761,726 and US Pat. Publ. Nos. 2017/0105763 and 2018/0125465 which disclose various devices capable of accessing the IO space.

In addition, commercially available systems for placing needles in the IO space include the Bone Injection Gun (Persys Medical), the EZ-IO (VidaCare Corp), and the FAST1 adult intraosseous infusion system (Pyng Medical Corp.). It is desirable to improve upon these state of the art devices and provide a device that is capable of accessing and administering a fluid to the IO space at a constant rate over an extended period of time.

### SUMMARY

According to one embodiment, the present disclosure provides a delivery device adapted to deliver a fluid into an IO space of a subject at a relatively constant volumetric flow rate over an extended period of time. The delivery device may include:

(i) an outer cartridge;

(ii) an inner cartridge configured to be movably disposed in a proximal direction within the outer cartridge and configured to hold a fluid container containing the fluid;

(iii) an actuator operable to activate the delivery device;

(iv) a plunger assembly sized and configured to be movably disposed within the fluid container from a first position to a second position;

(v) an energy storage member operable to produce a force to drive the plunger assembly from the first position to the second position after activation of the delivery device;

(vi) a releasable retainer member configured to secure the plunger assembly in the first position and to release the plunger assembly after activation of the delivery device; and

(vii) a fluid delivery system having an inlet configured to be in fluid communication with an outlet of the fluid container and an outlet configured to deliver the fluid contained in the fluid container to an IO access device or IV line positioned in the IO space of the subject

wherein in operation the delivery device is activated by activating the actuator to cause a sequence of movements whereby the inner cartridge is displaced within the outer cartridge in the proximal direction and the plunger assembly is subsequently released from the retainer member and displaced from the first position to the second position by the force produced by energy storage member to force the fluid confined in the fluid container to dispense through the fluid delivery system at the relatively constant volumetric flow rate to the IO access device or IV line and to the IO space of the subject.

According to another embodiment, the present disclosure provides a device for accessing an IO space of a subject and delivering fluid to the IO space of the subject at a relatively constant volumetric flow rate over an extended period of time. In this embodiment, the delivery device may include:

(i) an outer cartridge comprising an inner coupling end and configured to hold an aspiration container;

(ii) an inner cartridge configured to be movably disposed in a proximal direction within the outer cartridge and to hold a fluid container containing the fluid, the inner cartridge comprising an aspiration plunger movably coupled to its distal end sized and configured to be movably disposed within the aspiration container;

(iii) a first actuator operable to activate the delivery device;

(iv) a plunger assembly sized and configured to be movably disposed within the fluid container from a first position to a second position;

(v) an energy storage member operable to produce a first force to drive the plunger assembly from the first position to the second position after activation of the delivery device;

(vi) a releasable retainer member configured to secure the plunger assembly in the first position and to release the plunger assembly after activation of the delivery device;

(vii) an IO access device disposed in the outer cartridge and configured to access the IO space of the subject, the IO access device comprising a second actuator operable to activate the IO access device, a tissue penetrator assembly releasably coupled to the inner coupling end of the outer cartridge and a driver operable to produce a second force to drive the tissue penetrator assembly into the IO space after activation of the IO access device; and

(viii) a fluid delivery system configured to be fluidly coupled with the outlets of the aspiration container and fluid container and coupled to the tissue penetrator assembly.

According to yet another embodiment there is provided a system. The system may include a delivery device and an IO access device. The delivery device may include:

(i) an outer cartridge comprising an aspiration container;

(ii) an inner cartridge configured to be movably disposed in a proximal direction within the outer cartridge comprising a fluid container containing a medicament and an aspiration plunger movably coupled to its distal end sized and configured to be movably disposed within the aspiration container;

(iii) a first actuator operable to activate the delivery device;

(iv) a plunger assembly sized and configured to be movably disposed within the fluid container from a first position to a second position;

(v) an energy storage member operable to produce a first force to drive the plunger assembly from the first position to the second position after activation of the delivery device;

(vi) a releasable retainer member coupled to the plunger assembly to hold the plunger assembly in the first position and configured to release the plunger assembly after activation of the delivery device; and

(vii) a catch mechanism. The IO access device is configured to be securely attached to the delivery device and may include:

(i) a housing comprising an inner coupling end;

(ii) a second actuator operable to activate the IO access device;

(iii) a tissue penetrator assembly disposed within the housing and coupled to the coupling end of the housing where the tissue penetrator assembly is configured to be released from the coupling end after activation of the IO access device;

(iv) a driver operable to produce a second force to drive the tissue penetrator assembly into the IO space after activation of the IO access device;

(v) a fluid delivery system coupled to the tissue penetrator assembly and configured to be fluidly coupled to the outlets of the aspiration container and the fluid container of the delivery device; and

(vi) an engaging lock configured to be coupled to the catch mechanism of the delivery device to secure the IO access device to the delivery device.

In still yet another embodiment, the present disclosure provides a kit comprising the delivery device described above and a fluid container containing a medicament. In other embodiments, the kit may further include an IO access device disposed within the delivery device or separate from the delivery device but configured to be securely attached to the delivery device.

According to another embodiment, there is provided a system adapted to deliver a fluid into an IO space of a subject at a relatively constant volumetric flow rate over an extended period of time. The system generally includes:

(a) a delivery device including:

(i) a fluid container having an open proximal end and a closed distal end with an orifice, the fluid container further containing the fluid;

(ii) a flow regulator comprising an inlet in fluid communication with the fluid container and an outlet;

(iii) a fluid channel in fluid communication with the outlet of the flow regulator,

(iv) a plunger assembly positioned at or near the proximal end of the fluid container and sized and configured to be movably disposed within the fluid container from a first position to a second position; and

(v) an energy storage member operable to produce a force on the plunger assembly to move the plunger assembly from the first position to the second position once activated;

(b) an IO access device comprising:

(i) a removable actuator,

(ii) a driver configured to be movably disposed from a first position to a second position when the actuator is activated,

(iii) a tissue penetrator assembly coupled to the driver and configured to access the IO space when the driver moves from the first position to the second position,

(iv) a fluid channel assembly coupled to the tissue penetrator assembly and configured to place the fluid channel assembly in fluid communication with the fluid channel when the driver moves from the first position to the second position, and

(c) an outer cartridge housing the fluid delivery system and substantially all of the IO access device

wherein in operation the IO access device is activated by activation of the actuator which moves the driver from the first position to second position allowing the tissue penetrator assembly to access the IO space and place the fluid channel in fluid communication with the fluid channel which activates the energy member to provide the force to move the plunger assembly from the first position to the second position within the fluid container and dispense the fluid through the fluid channel to the fluid channel assembly and to the IO space at the relatively constant volumetric flow rate.

### BRIEF DESCRIPTION OF THE DRAWINGS

FIG. 1A is a cross-sectional view of a delivery device according to one embodiment in a first configuration;

FIG. 1B is a cross-sectional view of the delivery device of FIG. 1A in a second configuration;

FIG. 1C is a perspective view of the third fluid conduit of the delivery device of FIG. 1A attached to an intraosseous access device positioned at a desired target site of a subject;

FIG. 1D is a side view of the delivery device of FIG. 1A positioned on the skin of a subject;

FIG. 2 is a perspective view of a delivery device according to another embodiment;

FIG. 3 is an exploded view of the delivery device of FIG. 2;

FIG. 4 is a cross-sectional view of the outer cartridge of the delivery device of FIG. 2;

FIG. 5 is a cross-sectional view of the inner cartridge of the delivery device of FIG. 2;

FIG. 6 is a perspective view of the fluid delivery system of the delivery device of FIG. 2;

FIGS. 7A and 7B are cross-sectional views rotated 90 degrees of each other of the delivery device of FIG. 2 in a storage configuration;

FIGS. 8A and 8B are cross-sectional views rotated 90 degrees from each other of the delivery device of FIG. 2 in a first configuration;

FIGS. 9A and 9B are cross-sectional views rotated 90 degrees from each other of the delivery device of FIG. 2 in a second configuration;

FIG. 10A is a cross-sectional view of the delivery device of FIG. 1A further including an IO access device according to one embodiment, the IO access device in a first configuration;

FIG. 10B is a cross-sectional view of the delivery device of FIG. 10A with the IO access device in a second configuration;

FIG. 10C is a cross-sectional view of the delivery device of FIG. 10A where the IO access device and delivery device are each in a second configuration;

FIG. 10D is a side view of the delivery device and IO access device of FIG. 10A positioned on the skin of a subject;

FIG. 11 is a perspective view of a delivery device with an IO access device according to another embodiment;

FIG. 12 is an exploded view of the delivery device according to FIG. 11;

FIG. 13 is a cross-sectional view of the outer cartridge of the delivery device of FIG. 11;

FIG. 14 is a cross-sectional view of the inner cartridge of the delivery device of FIG. 11;

FIG. 15 is a perspective view of the fluid delivery system of the delivery device of FIG. 11;

FIG. 16A is a cross-sectional view of the delivery device of FIG. 11 where the IO access device is in a first configuration and the delivery device is in an initial position;

FIG. 16B is a cross-sectional view of the delivery device of FIG. 11 where the IO access device is in a second configuration;

FIG. 16C is a cross-sectional view of the delivery device of FIG. 11 where the delivery device is in a first configuration;

FIG. 16D is a cross-sectional view of the delivery device of FIG. 11 where the delivery device is in a second configuration;

FIG. 17A is a perspective view of a delivery device and IO access device according to one embodiment;

FIG. 17B is a perspective view of the delivery device coupled to the IO access device of FIG. 17A;

FIGS. 18 and 18A are exploded views of the delivery device and IO access device of FIG. 17A;

FIGS. 18B to 18E are perspective views of the inner cartridge, IO access device, and outer cartridge of the delivery device and IO access device of FIG. 17A;

FIG. 19A is a cross-sectional view of a delivery device according to another embodiment;

FIG. 19B is a cross-sectional view of the delivery device according to FIG. 19A in a first configuration; and

FIG. 19C is a cross-sectional view of the delivery device according to FIG. 19A in a second configuration.

FIG. 20 is a perspective view of a delivery device and IO access device according to one embodiment.

FIG. 21 is an exploded view of the delivery device and IO access device of FIG. 20.

FIG. 22A is a top plan view of the outer cartridge of FIG. 20.

FIG. 22B is a cross-sectional view of the outer cartridge in FIG. 22A, viewed in the direction of A-A shown in FIG. 22A.

FIG. 23A is top view of the second actuator of the IO access device of FIG. 20.

FIG. 23B is a cross-sectional view of the second actuator of FIG. 23A, viewed in the direction of A-A shown in FIG. 23A.

FIG. 23C is a side view of the second actuator of FIG. 23A.

FIG. 23D is a cross-sectional view of the second actuator of FIG. 23A, viewed in the direction of B-B shown in FIG. 23C.

FIGS. 24A and 24B are cross-sectional views of the delivery device and IO access device of FIG. 21 in a first configuration.

FIG. 25A is a side view of the delivery device and IO access device of FIG. 21 in a second configuration.

FIG. 25B is a cross-sectional view of the delivery device and IO access device of FIG. 21 in a second configuration, viewed in the direction of A-A shown in FIG. 25A.

FIG. 26A is a side view of the delivery device and IO access device of FIG. 21 in a third configuration.

FIG. 26B is a cross-sectional view of the delivery device and IO access device of FIG. 21 in a second configuration, viewed in the direction of A-A shown in FIG. 26A.

FIG. 27 is a cross-sectional view of the delivery device of FIG. 21 in a fourth configuration.

FIGS. 28A and 28B are cross-sectional views of part of the IO access device of FIG. 21 in a fifth configuration (only fluid channel assembly remaining).

### DETAILED DESCRIPTION

The following terms shall have the following meanings:

The term “comprising” and derivatives thereof are not intended to exclude the presence of any additional component, step or procedure, whether or not the same is disclosed herein. In contrast, the term, “consisting essentially of” if appearing herein, excludes from the scope of any succeeding recitation any other component, step or procedure, except those that are not essential to operability and the term “consisting of”, if used, excludes any component, step or procedure not specifically delineated or listed. The term “or”, unless stated otherwise, refers to the listed members individually as well as in any combination.

As used herein, the term “about” generally means plus or minus 10% of the value stated. For example, about 0.5 would include 0.45 to 0.55.

The articles “a” and “an” are used herein to refer to one or to more than one (i.e. to at least one) of the grammatical objects of the article. By way of example, “a container” means one container or more than one container. The phrases “in one embodiment”, “according to one embodiment” and the like generally mean the particular feature, structure, or characteristic following the phrase is included in at least one embodiment of the present disclosure, and may be included in more than one embodiment of the present disclosure. Importantly, such phrases do not necessarily refer to the same aspect. If the specification states a component or feature “may”, “can”, “could”, or “might” be included or have a characteristic, that particular component or feature is not required to be included or have the characteristic.

As used in this specification and the appended claims, the words “proximal” and “distal” refer to directions closer to and away from, respectively, an operator (e.g., surgeon, physician, nurse, technician, etc.) of the delivery device.

The term “intraosseous space” or “IO space” refers to the spongy, cancellous bone of the epiphysis and the medullary cavity of the diaphysis, which are connected.

The term “intraosseous access device” or “IO access device” includes, but not limited to, any actuator, hollow needle, hollow drill bit, tissue penetrator assembly, fluid channel assembly, bone penetrator, catheter, cannula, trocar, inner penetrator, outer penetrator, IO needle or IO needle set and a driver operable to provide access to an IO space or interior portions of a bone. A wide variety of trocars, spindles and/or shafts may be disposed within a cannula during insertion at a selected insertion site. Such trocars, spindles and shafts may also be characterized as inner penetrators. A catheter, cannula, hollow needle or hollow drill bit may sometimes be characterized as an outer penetrator.

The term “fluid” includes any liquid, such as but not limited to, blood, water, saline solutions, IV solutions or plasma, or any mixture of liquids, particulate matter, medicament, dissolved medicament and/or drugs appropriate for injection into the IO space of a subject.

The term “container” refers to a pharmaceutically acceptable container comprising a chamber suitable to house a fluid or air. Containers can include, but are not limited to vials, barrels, ampoules or bottles and in some embodiments are made of glass, plastic, composites, laminates or metal.

The term “kit” may be used in this application to describe a wide variety of bags, containers, carrying cases and other portable enclosures which may be used to carry and store delivery devices, IO access devices and/or intravenous devices of the present disclosure along with related components and accessories. Such kits and their contents along with applicable procedures may be used to provide access to a subject's vascular system in accordance with teachings of the present disclosure.

As used herein, a “subject” may be a human or non-human mammal. Non-human mammals include, for example, livestock and pets, such as ovine, bovine, porcine, canine and feline mammals. Preferably, the subject is a human.

The term “relatively constant” as used herein is a broad term, and is to be given its ordinary and customary meaning to a person of ordinary skill in the art (and is not to be limited to a special or customized meaning), and refers without limitation to a value that, although it may change with time, does not change so substantially so as to render the value inoperative or substantially less operative. For example, maintaining a standard deviation of to within 3% or up to 5% of the mean for an hour would be considered relatively constant with respect to a volumetric flow rate.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

Embodiments of the present disclosure are generally directed to a delivery device adapted to deliver fluid into the IO space of a subject at a relatively constant volumetric flow rate over an extended period of time (i.e. at least 1 minute). In some embodiments, the fluid may be delivered to the IO space of the subject at a volumetric flow rate of about 0.5 mL/minute or about 0.6 mL/minute or about 0.7 mL/minute or about 0.8 mL/minute or about 0.9 mL/minute, or about 1.0 mL/minute, or about 1.1 mL/minute, or about 1.2 mL/minute, or about 1.3 mL/minute, or about 1.4 mL/minute, or about 1.5 mL/minute. In still other embodiments, the fluid may be delivered to the IO space of the subject at a volumetric flow rate of up to about 2 mL/min, or up to about 3 mL/min, or up to about 4 mL/min, or up to about 5 mL/min, or up to about 6 mL/min, or up to about 7 mL/min or up to about 8 mL/min or up to about 9 mL/min or up to about 10 mL/min. In other embodiments, the fluid may be delivered to the IO space of the subject at a volumetric flow rate within a range of about 0.7 mL/minute to about 1.3 mL/minute, or about 0.9 mL/minute to about 1.1 mL/minute.

In some embodiments, the delivery device may also be combined with or include an IO access device configured for insertion into the IO space and which is coupled to or configured to be coupled to or which can be decoupled from the delivery device after the IO space has been accessed by the IO access device. In other embodiments, the delivery device may be configured to fluidly connect to an IO access device already inserted at a desired area of the subject. IO access may be used as a bridge during emergency conditions until conventional IV sites can be found and used. Conventional IV sites often become available because fluids and/or medication provided via IO access may stabilize a subject and expand veins and other portions of a subject's vascular system. Accordingly, in other embodiments, the delivery device may be configured to fluidly connect to a conventional IV line already attached at a desired area of the subject.

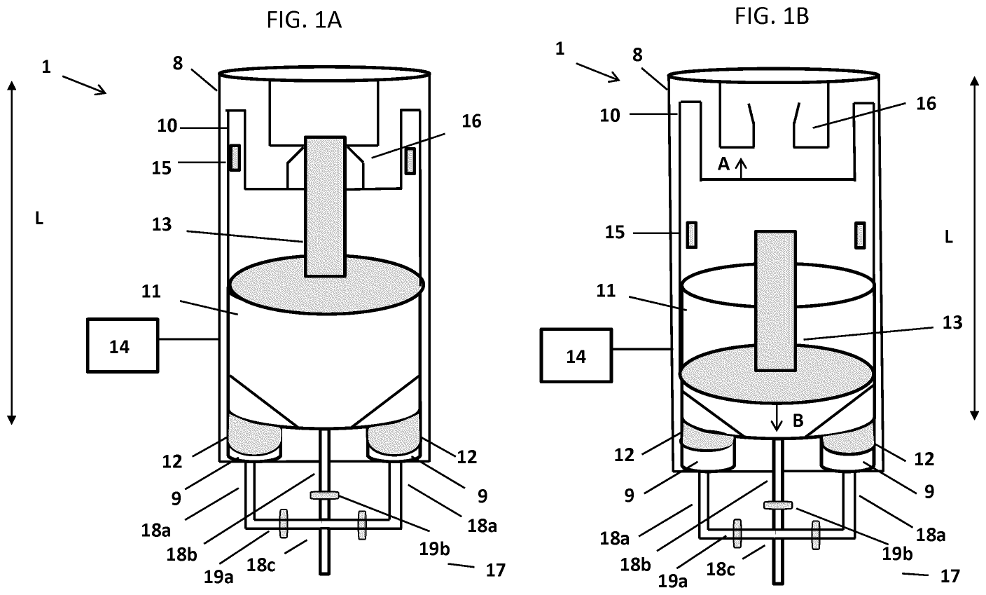

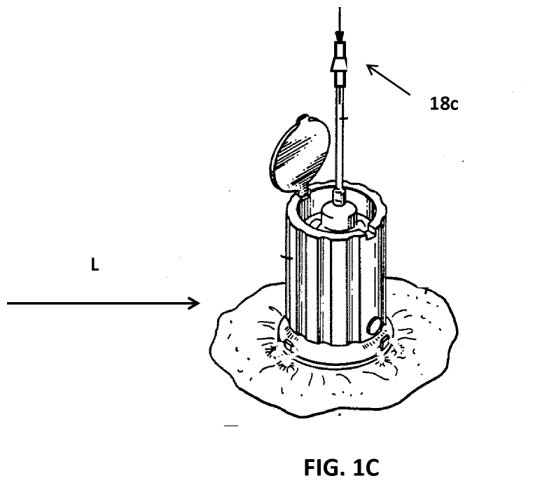

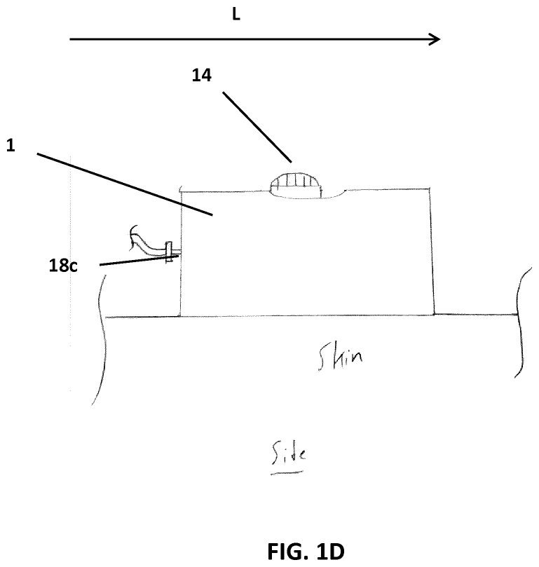

Turning now to the drawings and in particular, to FIGS. 1A and 1B, a cross-sectional view of a delivery device 1 is shown according to one embodiment in a first configuration and in a second (advanced) configuration, respectively. In general terms, the delivery device 1 includes an outer cartridge 8. The outer cartridge 8 is configured in some embodiments to hold an aspiration container 9 (in the embodiment shown in FIGS. 1A and 1B, two aspiration containers 9 are depicted, however other embodiments may include one aspiration container 9 or three or more aspiration containers 9). The aspiration container 9 has an open proximal end and a closed distal end comprising an orifice or outlet.

The delivery device 1 also includes an inner cartridge 10, configured to be movably disposed within the outer cartridge 8, and may comprise an aspiration plunger 12 movably coupled to its distal end. The inner cartridge 10 is further configured to hold a fluid container 11. The fluid container 11 has an open proximal end configured to receive a plunger assembly 13 and a closed distal end comprising an orifice or outlet. The plunger assembly 13 is configured to be movably disposed within the fluid container 11. The fluid container 11 contains (i.e., is filled or partially filled with) a fluid.

The delivery device 1 also includes an energy storage member 15 operable to drive the plunger assembly 13 in a distal direction, an actuator 14 operable to activate the delivery device 1, a releasable retainer member 16 configured to secure the plunger assembly 13 and to release the plunger assembly 13 after activation of the delivery device 1 and a fluid delivery system 17 having an inlet configured to be in fluid communication with the outlet of the fluid container and the outlet of the aspiration container and an outlet configured to be fluidly connected to an IO access device or IV line positioned within the IO space of the subject. The fluid delivery system 17 may comprise a first conduit 18a fluidly coupled to the orifice or outlet of the aspiration container 9 and a second conduit 18b fluidly coupled to the orifice or outlet of the fluid container 11, and one-way valves 19a and 19b. The outlets of the one way valves 19a and 19b are fluidly coupled to form a third conduit 18c. First, second and third fluid conduits 18a, 18b and 18c may be sterile flexible tubing. Third conduit 18c in some embodiments will have just enough slack to uncoil and become taut once fluidly coupled to an IO access device.

The delivery device 1 can generally be operated by activating the actuator 14 to cause a sequence of movements whereby the inner cartridge 10, along with aspiration plunger 12, are displaced within the outer cartridge 8 in a proximal direction to aspirate third fluid conduit 18c and the plunger assembly 13 is subsequently released from the releasable retainer member 16 and displaced in a distal direction within the fluid container 11 by the energy storage member 15 to force the fluid confined in the fluid container 11 through the second tubular port to the second and third fluid conduits 18b and 18c at a relatively constant volumetric flow rate. The distal end of the third conduit 18c may be fluidly connected to an IO access device implanted into the IO space of a subject as shown in FIG. 1C or an IV line (not shown). For ease of administration and attachment to the IO access device or IV line, in one embodiment, the delivery device 1 can be positioned on the skin of the subject as shown in FIG. 1D and movement of the components of the delivery device 1 occur in the direction L (i.e. parallel to the skin of the subject).

The actuator 14 may be any suitable device for activating the delivery device 1, such as, for example, a handle, a lever, a push button, a slidable button or a trigger. Upon activation, the inner cartridge 10 is displaced within the outer cartridge 8, as indicated by arrow A in FIG. 1B, along a longitudinal axis L between a first position (FIG. 1A) and a second position (FIG. 1B). As illustrated, when the inner cartridge 10 is displaced in a proximal direction from the first position to second position, the aspiration plunger 12 is also moved in the proximal direction from a first position to second position within the aspiration container 9 thereby drawing fluid and/or air contained within third conduit 18c into the aspiration container 9. One-way valve 19b is configured to completely prevent fluid and/or air within the third conduit 18c from being drawn into the second conduit 18b and one way valve 19a is configured to completely prevent the backflow of any fluid and/or air that may be contained within or drawn into the outer containers 9.

The releasable retainer member 16 is disposed within the interior of delivery device 1 and is also configured to selectively deploy the energy storage member 15 from its first configuration to second configuration after the inner cartridge 10 has been displaced in the proximal direction from its first position to second position. The releasable retainer member 16 can be any suitable mechanism for releasably retaining the plunger assembly 13 and deploying the energy storage member 15, such as, for example, a mechanical linkage, a spring-loaded rod, a tensioned latch or tab or the like. In this manner, the operator can actuate the plunger assembly 13 by manipulating a portion of the releasable retainer member 16.

In some embodiments, the fluid in fluid container 11 comprises a medicament, such as, for example, tranexamic acid or any of the other medicaments further described below. As illustrated, the plunger assembly 13 may be moved in a distal direction, as indicated by arrow B in FIG. 1B, along a longitudinal axis L between a first position (FIG. 1A) and second position (FIG. 1B) by energy storage member 15. When the plunger assembly 13 is in its first (or retracted) position, the plunger assembly 13 is positioned at the proximal end of inner cartridge 10. In this first position, the plunger assembly 13 may be slightly spaced apart from fluid contained within fluid container 11, or in some embodiments in fluid communication with the fluid contained within fluid container 11. When the plunger assembly 13 is displaced (or advanced) to the second position, the plunger assembly 13 is advanced from the proximal end of the inner cartridge 10 towards the distal end of the fluid container 11. In this manner, as the plunger assembly 13 is moved (or advanced) between the first and second positions, fluid is conveyed from within fluid container 11 into the IO space of a subject via the second conduit 18b and third conduit 18c at a relatively constant volumetric flow rate.

The energy storage member 15 can be any device for storing energy. Thus, the energy storage member 15 may be a mechanical energy storage member, such as a spring, a device containing compressed gas, a device containing a vapor pressure-based propellant or the like. In other embodiments, the energy storage member 15 can be an electrical energy storage member, such as a battery, a capacitor, a magnetic energy storage member or the like. In yet other embodiments, the energy storage member 15 can be a chemical energy storage member, such as a container containing two substances that, when mixed, react to produce energy.

As shown, the energy storage member 15 also defines longitudinal axis L. The energy storage member 15 can be moved within the inner cartridge 10 along the longitudinal direction L between a first configuration (FIG. 1A) and a second configuration (FIG. 1B). When the energy storage member 15 is in its first configuration, the energy storage member 15 has a first potential energy. When the energy storage member 15 is in its second configuration, the energy storage member 15 has a second potential energy that is less than the first potential energy. The energy storage member 15 is operably connected to the plunger assembly 13 such that when the energy storage member 15 moves from its first configuration to its second configuration, it converts at least a portion of its first potential energy into kinetic energy to move the plunger assembly 13 between its first position and second position. Said another way, the movement of the energy storage member 15 from its first configuration to its second configuration results in the production of a force that acts upon the plunger assembly 13 to move the plunger assembly 13 between its first position and second position and thereby dispense fluid contained within the fluid container 11 through second conduit 18b and third conduit 18c at a relatively constant volumetric flow rate. A desired flow of fluid (i.e. relatively constant volumetric flow rate) that is being dispensed into the second conduit 18b can be achieved by matching the force produced by the energy storage member 15 with a particular size diameter of the orifice of fluid container 11. For example, if the energy storage member 15 is a spring, one can ensure that a desired flow of fluid is being dispensed through the orifice of the fluid container 11 by matching the diameter of the orifice or outlet of the fluid container with a spring constant of the spring. In another embodiment, the desired flow of fluid that is being dispensed into the second conduit 18b from the fluid container 11 can be achieved using a flow regulator, such as a variable adjustable valve positioned between the orifice or outlet of the fluid container 11 and one-way valve 19b or a helical channel with a diaphragm positioned at the distal end that can be closed then opened by a pressure differential caused by movement of the plunger assembly. In another embodiment, a plate having a particular size orifice (i.e. orifice plate) positioned between the outlet of fluid container 11 and one-way valve 19b may be used to achieve the desired flow of fluid that is being dispensed into fluid conduit 18b.

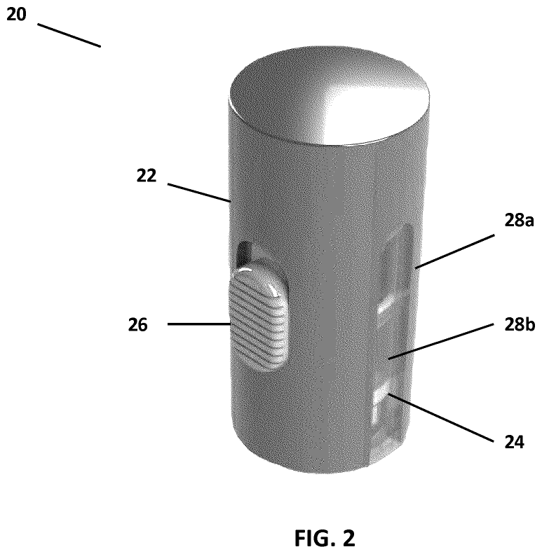

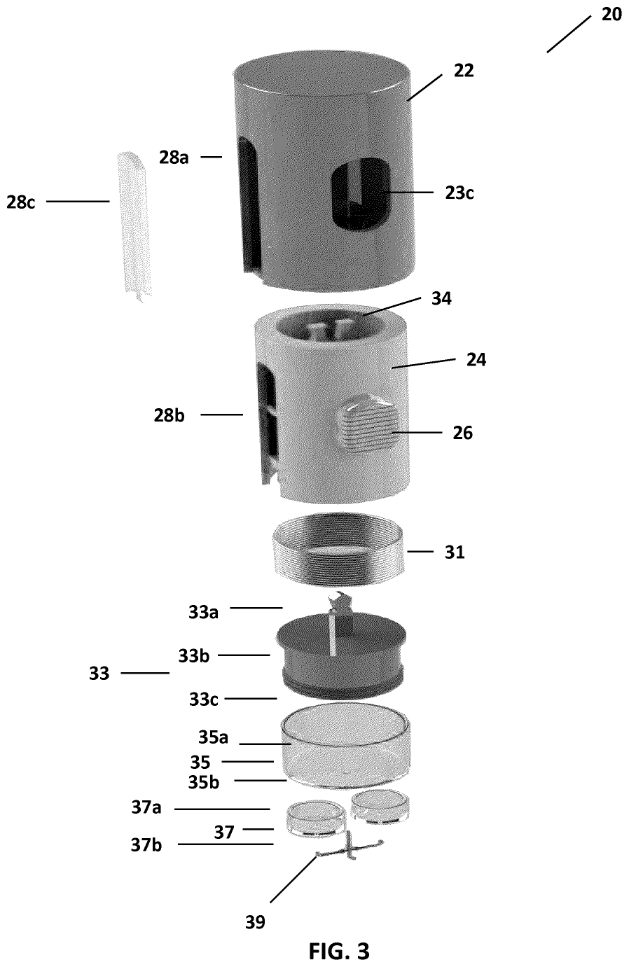

With reference now to FIGS. 2 through 6, a delivery device according to an embodiment is shown and generally designated by reference numeral 20. As shown in FIGS. 2 and 3, the delivery device 20 includes an outer cartridge 22, an inner cartridge 24 configured to be movably disposed within the outer cartridge 22, an actuator 26, first and second elongated windows 28a, 28b configured to allow an operator to view the contents within the delivery device 20, an energy storage member 31, a plunger assembly 33, a releasable retainer member 34, a fluid container 35, an aspiration container 37 and a fluid delivery system 39. In some embodiments, the windows 28a, 28b may be open, with no material or they may comprise a clear material 28c, such as a translucent or transparent material, to allow the operator to view the contents of the fluid container 35 within the delivery device 20.

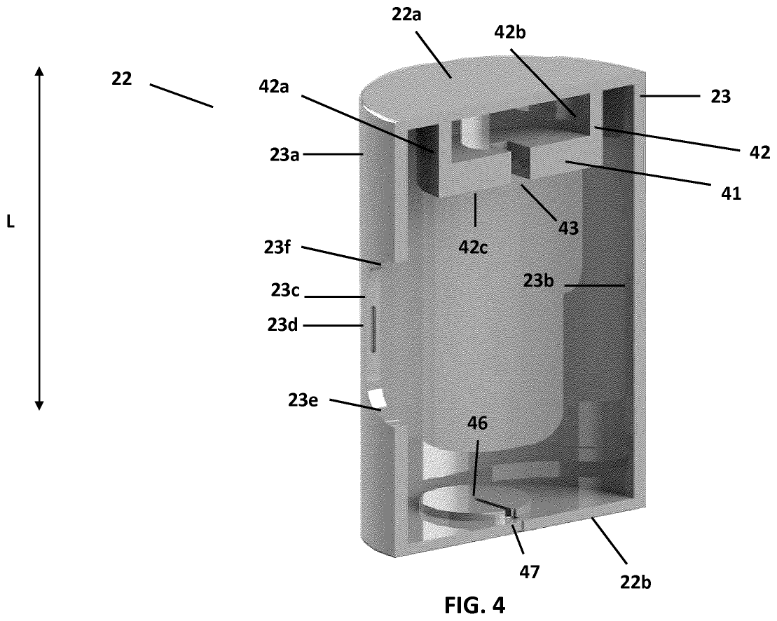

With reference now to FIGS. 3 and 4, the outer cartridge 22 is generally shaped and dimensioned to fit within an operator's hand and includes a wall 23 with an exterior surface 23a and an interior surface 23b. While a unitary (i.e., one-piece) wall 23 has been illustrated in FIG. 4 that defines both the exterior and interior surfaces 23a, 23b, it will be understood that according to other embodiments the wall 23 may include a plurality of layers with different layers defining the exterior and interior surfaces 23a, 23b. Wall 23 may further include an opening 23c to accommodate actuator 26 of inner cartridge 24 which is operated by the operator. Opening 23c is configured to allow actuator 26 to be movably displaced in the longitudinal direction L due to the presence of two opposing rails 23d positioned on the walls of the opening 23c. The rails 23d lead into an internal distal stop end 23e and an internal proximal stop end 23f limiting the distance the actuator 26 can travel in the L direction within the opening 23c. In some embodiments, the rails 23d may have a length of between about 5 mm to about 10 mm.

According to certain embodiments of the present disclosure, the wall 23 may be rigid. According to other embodiments, the wall 23 may be flexible, whether according to the nature of the material that defines the wall 23 or according to the nature of the structure of the wall 23. The wall 23 may be made of glass, metal, or polymer, for example. In particular, polymer versions may be made of polycarbonate, polypropylene, polyethylene (such as high density polyethylene), polytetrafluoroethylene, cyclic olefin polymer, cyclic olefin copolymer, crystal zenith olefinic polymer, nylon, or engineering resins. As to flexible versions of the wall 23, butyl rubber, silicon-based rubber, latex-based rubber, coated rubber, as well as multi-layer polymer films, such as polyethylene (such as low density polyethylene) and polypropylene, may be used.

The wall 23 may have a generally cylindrical shape (although other shapes are contemplated, such as rectangular). According to some embodiments, the wall 23 may have a length of between about 70 mm to about 140 mm and defines two opposed, closed ends, proximal end 22a and distal end 22b, each having a diameter of between about 20 mm to about 50 mm. Attached to the interior surface of proximal end 22a is an inner cylindrical wall 42 having an exterior surface 42a and an interior surface 42b. Inner cylindrical wall 42, which in some embodiments may have a length of between about 10 mm to about 30 mm, defines an inner cylindrical cross-section having a closed distal end 41 comprising a cutout section 43. The closed distal end 41 therefore has a diameter that is less than the diameter for proximal end 22a and distal end 22b of wall 23. In some embodiments, the diameter of the closed distal end 41 ranges between about 20 mm to about 40 mm. The cutout section 43 is shaped and configured to engage the retainer member 34 (not shown) when the inner cartridge 24 is in the first position. For instance, the wall of the cutout section 43 may be sized and adapted to correspondingly mate with the shape of the retainer member 34 when the inner cartridge 24 in in its first position and to allow plunger assembly 33 to be movably disposed therein when the inner cartridge 24 is moved to its second position as further described below.

The exterior surface 42a of inner cylindrical wall 42 and the interior surface 23b of wall 23 are spaced apart a sufficient distance to allow the inner cartridge 24 to be movably disposed therein in a longitudinal direction L when the actuator 26 is activated.

An aspiration container mount 46 is attached to the interior surface of the distal end 22b and may be formed of the same material as the wall 23 or a different rigid solid material, such as plastic. The aspiration container mount 46 is sized and adapted to hold the aspiration container 37 (not shown) in alignment with an aspiration plunger (not shown) of inner cartridge 24. The aspiration container mount 46 further includes a notch 47 sized and adapted to receive and hold first conduit 18a.

The delivery device 20 is configured to include an aspiration container 37 disposed within the outer cartridge 22. The aspiration container 37 can be positioned on or held by the aspiration mount 46. In some embodiments, the aspiration container 37 may have a length of about 4 mm to about 12 mm. The aspiration container 37 includes an open proximal end 37a and a closed distal end 37b, each with a diameter of between about 12 mm to about 16 mm when two aspiration containers are present or between about 20 mm to about 50 mm when one aspiration container is present. The proximal end of the aspiration container 37 is sized and adapted to receive the aspiration plunger (not shown) of inner cartridge 24. The closed distal end 37b includes an orifice fluidly coupled to first conduit 18a, such as by a luer connector, threads, a snap-fit, a latch, a lock, a friction fit coupling, or any other suitable coupling features. The orifice at the closed distal end of the aspiration container 37 may have a diameter of between about 0.159 mm to about 1.6 mm. In some embodiments, the aspiration container 37 can be, for example, a glass vial.

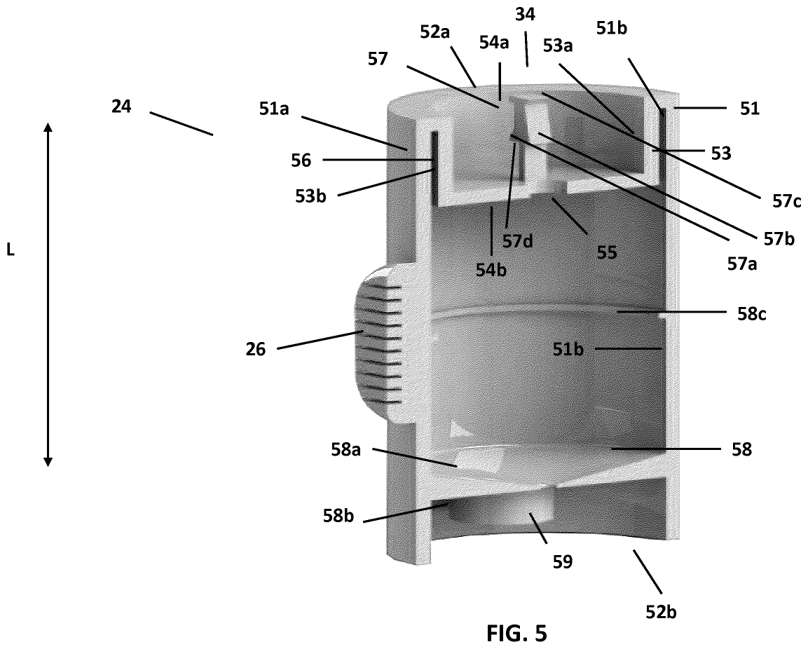

With reference now to FIGS. 3 and 5, inner cartridge 24 includes a wall 51 with an exterior surface 51a and an interior surface 51b. Similar to outer cartridge 22, while a unitary (i.e., one-piece) wall 51 has been illustrated in FIG. 5 that defines both the exterior and interior surfaces 51a, 51b, it will be understood that according to other embodiments the wall 51 may include a plurality of layers with different layers defining the exterior and interior surfaces 51a, 51b. Accordingly, wall 51 may be made of glass, metal, or polymer, for example. In particular, polymer versions may be made of polycarbonate, polypropylene, polyethylene (such as high density polyethylene), polytetrafluoroethylene, cyclic olefin polymer, cyclic olefin copolymer, crystal zenith olefinic polymer, nylon, or engineering resins.

Wall 51 is sized and configured to be movably disposed within outer cartridge 22 and thus may have a correspondingly similar, but smaller shape as the outer cartridge 22, such as a generally cylindrical (or rectangular) shape. According to some embodiments, wall 51 may have a length of between about 60 mm to about 140 mm and may define two opposed ends, a semi-enclosed proximal end 52a and an open distal end 52b. The semi-enclosed proximal end 52a includes an inner cylindrical wall 53, which may be rigid or flexible, attached to its interior surface. Inner cylindrical wall 53, which in some embodiments may have a length of between about 25 mm to about 45 mm, has an interior surface 53a and an exterior surface 53b and defines two opposed ends, an open proximal end 54a and a closed distal end 54b comprising a cutout section 55. The cutout section 55 is shaped and configured to prevent the plunger rod 33a (not shown) of the plunger assembly 33 from being movably disposed within the inner cartridge 24 when the inner cartridge 24 is in the first position and be movably disposed in the longitudinal direction L after the actuator 26 has been activated. For instance, the wall of the cutout section 55 may be sized and adapted to correspondingly mate with the plunger assembly 33 (not shown) when the inner cartridge 24 is in the first position. For instance, the wall of the cutout section 55 may be sized and adapted to correspondingly mate with the shape of the plunger assembly 33 when the inner cartridge 24 in in its first position and to allow plunger assembly 33 to be movably disposed therein when the inner cartridge 24 is moved to its second position as further described below.

The exterior surface 53b of inner cylindrical wall 53 and the interior surface 51b of wall 51 are spaced apart to form an inner housing 56. Inner housing 56 is sized and adapted to allow the energy storage member 31 (not shown) to be movably disposed therein from its first configuration to second configuration after activation.

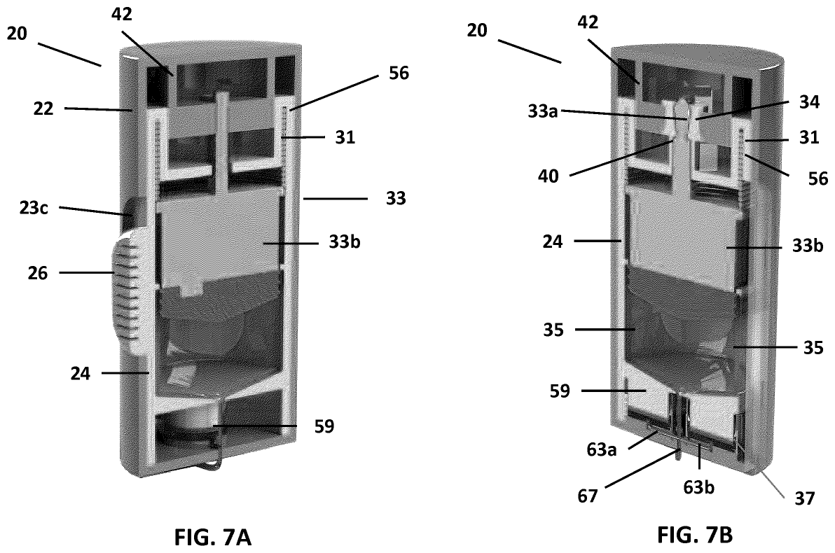

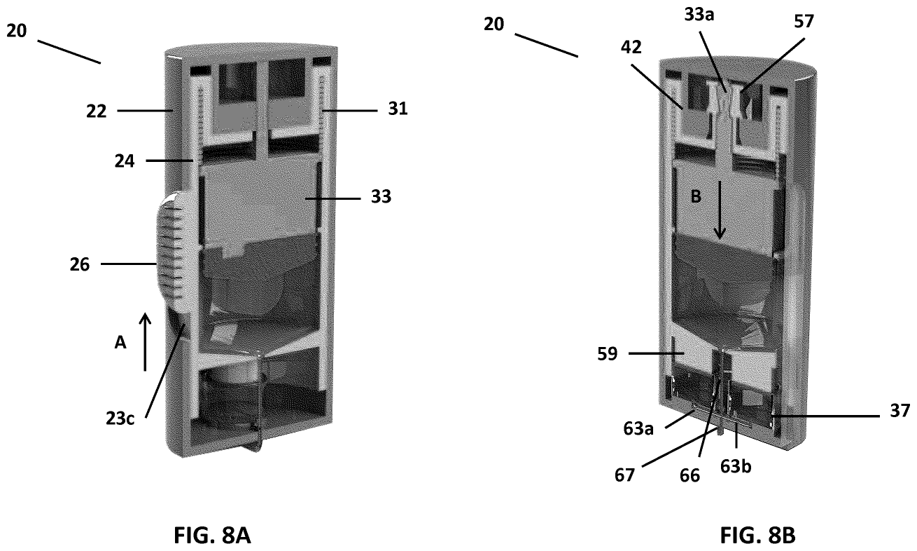

Actuator 26, in the form a slide button, is attached to the exterior surface 51a of wall 51. The top surface of actuator 26 is ribbed in a direction perpendicular to the direction of actuator movement, as shown, to allow the operator's finger to engage the actuator 26 without slipping. The actuator 26 is initially in the position shown in FIG. 7A and is advanced in the proximal direction (i.e., in the direction towards the proximal end 22b of the outer cartridge 22) to its second position shown in FIG. 8A by the operator during operation of the device 20. As will be described in detail below, movement of the actuator 26 from its first position to second position activates the delivery device 20 to cause a sequence of movements of the components within the delivery device that subsequently lead to the delivery of fluid contained within the delivery device.

The releasable retainer member 34, shown in the form of one or more latch tabs 57, is fixedly attached to the interior surface of distal end 54b of inner cylindrical wall 53. The latch tabs 57 have a downwardly facing outward shoulder 57a, a downwardly facing inward shoulder 57b an upper flange 57c and a lower flange 57d. The latch tabs 57 are inherently biased outwardly, with the wall of the cutout section 43 of outer cartridge 22 being shaped and configured so as to interferingly engage downwardly facing outward shoulder 57a under force of movement of the energy storage member 31 when the delivery device 20 is in an initial position. In addition, downwardly facing inward shoulder 57b and lower flange 57c are shaped and configured to interferingly engage the plunger rod 33a (not shown) of the plunger assembly 33 when the delivery device 20 is in the initial position. With this arrangement, movement of the plunger assembly 33 in the L direction is limited due to inter engagement between the downwardly facing outward shoulder 57a of the latch tabs 57 and the cutout section 43 of outer cartridge 22 as well as by the inter engagement between downwardly facing inward shoulder 57b and lower flange 57d of the latch tabs 57 and the plunger rod 33a of plunger assembly 33. The plunger assembly 33 is thus retained before use. Once the inner cartridge 24 has moved in a proximal direction between its first position and second position (i.e. upon activation of actuator 26), latch tabs 57 will also have moved to a position within the inner cylindrical cross section defined by inner cylindrical wall 42 of outer cartridge 22 thus allowing the latch tabs 57 to spring outwards and thereby releasing the plunger assembly 33 from engagement. Additionally, once the latch tabs 57 have moved to the position within the inner cylindrical cross section defined by inner wall 42, lower flange 57d of the latch tabs 57 will now be in inter engagement with the upper surface of distal end 42c of the inner cylindrical wall 42 thereby preventing the inner cartridge 24 from being displaced in the distal direction (see FIG. 8B).

The inner cartridge 24 also includes a fluid container mount 58 sized and adapted to hold fluid container 35. The fluid container mount 58 includes an upper surface 58a for engaging the distal end of the fluid container 35 and a lower surface 58b. The fluid container mount 58 also includes an upper ring portion 58c to prevent the fluid container from moving proximally. An aspiration plunger 59 is movably coupled to the lower surface 58b of fluid container mount 58. The aspiration plunger 59 is sized and adapted to engage the inner wall of the aspiration container 37 but be movably disposed within the aspiration container 37 when the inner cartridge 24 is moved from its first position to second position. In embodiments, aspiration plunger 59 may or may not include a plug (not shown) positioned at its distal end to engage the inner sidewalls of the aspiration container 37.

The delivery device 20 is therefore configured to include a fluid container 35 disposed within the inner cartridge 24 and held by the fluid container mount 58. The fluid container 35 contains (i.e., is filled or partially filled with) a fluid. In some embodiments the amount of fluid container 35 is in the range of about 1 millilitre (mL) to about 20 mL. The fluid container 35 includes an open proximal end 35a and a closed distal end 35b comprising an orifice that is fluidly coupled to the fluid conduit 18b (not shown), such as by a luer connector, threads, a snap-fit, a latch, a lock, a friction fit coupling, or any other suitable coupling features. The fluid container 35 may have a length of between about 10 mm to about 40 mm. The proximal end 35a may have a diameter of between about 25 mm to about 40 mm and the distal end 35b may have a diameter of between about 2 mm to about 3 mm. The orifice of the distal end 35b may have a diameter of between about 0.05 mm to about 1.6 mm.

In the embodiment shown in FIG. 3, the energy storage member 31 is a mechanical energy storage member comprising a spring, such as, for example, a helical, compression, extension, torsion, constant, variable, variable stiffness or any other type of spring having a spring constant ranging between about 1 N/m to about 500 N/m. The energy storage member 31 is operatively coupled to the plunger assembly 33 and produces a force on the plunger assembly 33 when the energy storage member 31 moves from its first configuration to second configuration after the actuator 26 has been activated.

As shown in FIG. 3, the plunger assembly 33 comprises a plunger rod 33a and plunger 33b. In some embodiments, the plunger assembly 33 may be formed as a single piece (as shown) or modular components in other embodiments. The modular components may be fixed to one another or located adjacently, but not connected, so as to move together.

A distal end portion of the plunger assembly 33 is configured to be disposed within fluid container 35. The distal end portion of the plunger assembly 33 can be coupled to and/or in contact with a plug 33c which is configured to be in fluid communication with the fluid disposed within an internal volume defined by the fluid container 35. The distal end portion of the plunger assembly 33 is configured to be movably displaced within the internal volume defined by the fluid container 35 due to the force produced by the energy storage member 31 when the energy storage member 31 moves from its first configuration to second configuration. In this manner, the energy storage member 31 acting on the plunger assembly 33 can displace the plug 33c within the fluid container 35 to expel the fluid through the orifice of distal end 35b of fluid container 35. The sidewalls of the plug 33c can be configured to contact the interior surfaces of the sidewalls of the fluid container 35 such that the plug 33c forms a fluid-tight seal with the sidewalls of the fluid container 35, for example, to prevent leakage of the fluid. The plug 33c can be made of an inert and/or biocompatible material which is rigid but soft. Example materials include rubber, silicone, plastic, polymers, any other suitable material or combination thereof. In some embodiments, the plug 33c can be monolithically formed with the plunger assembly 33.

The plunger rod 33a is attached to the proximal end of plunger 33b and includes an elongated lower member and a shaped upper member sized and configured to engage the downwardly facing inward shoulder 57b and lower surface of upper flange 57c of the latch tabs 57 when the plunger assembly 33 is in its first position. In the embodiment shown in FIG. 3, the shaped upper member of the plunger rod 33a has an upwardly pointing arrow-shape however any shape may be utilized so long as it engages with downwardly facing inward shoulder 57b and lower surface of upper flange 57c of the latch tabs 57. Plunger 33b is generally cylindrically shaped having a proximal end and a distal end and is configured to be movably disposed within the interior volume defined by the fluid container 35.

As described above, the fluid container 35 defines an internal volume configured to house a fluid. The fluid may comprise a medicament such as, but not limited to, an analgesic, anti-inflammatory agent, anthelmintic, anti-arrhythmic agent, antibiotic (including penicillin's), anticoagulant, antidepressant, antidiabetic agent, antiepileptic, antihistamine, antihypertensive agent, antimuscarinic agent, antimycobactefial agent, antineoplastic agent, antifibrinolytic, immunosuppressant, antithyroid agent, antiviral agent, anxiolytic sedative (hypnotics and neuroleptics), astringent, beta-adrenoceptor blocking agent, blood product and substitutes, cardiac inotropic agent, corticosteroid, cough suppressant (expectorants and mucolytics), diagnostic agent, diuretic, dopaminergic (antiparkinsonian agents), haemostatic, immunological agent, lipid regulating agent, muscle relaxant, parasympathomimetic, parathyroid calcitonin and biphosphonate, prostaglandin, radiopharmaceutical, sex hormone (including steroids), anti-allergic agent, stimulant and anorexic, sympathomimetic, thrombolytic, thyroid agent, PDE IV inhibitor, NK3 inhibitor, ppar agent, NK-2 inhibitor, CSBP/RK/p38 inhibitor, antipsychotics vasodilator and xanthine.

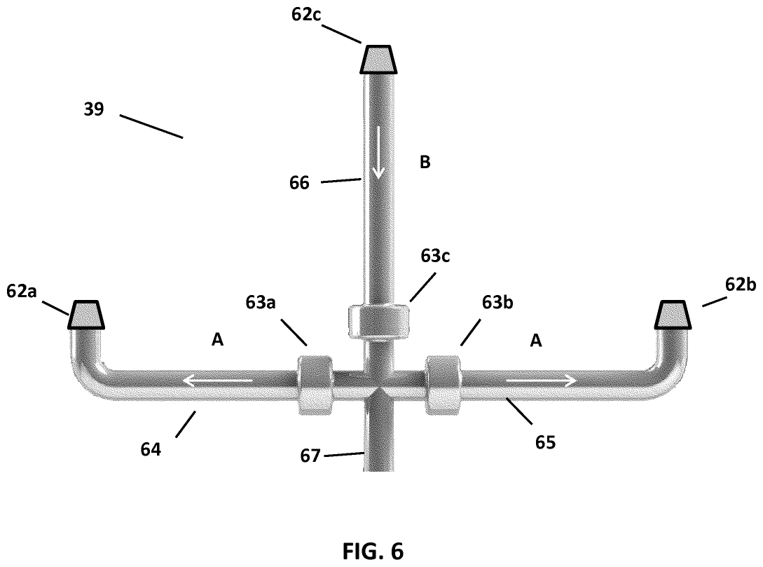

With reference now to FIGS. 3 and 6, delivery device 20 includes fluid delivery system 39 configured to fluidly couple to the orifice of the distal end 33b of fluid container 35 and to the orifice of the distal end 37b of aspiration container 37. In the FIG. 6 embodiment, the fluid delivery system 39 includes first, second and third tubular ports 62a, 62b and 62c. First and second tubular ports 62a, 62b are configured to fluidly couple with the orifices of the aspiration containers 37. Third tubular port 62c is configured to fluidly couple with the orifice of fluid container 35. Fluid delivery system 39 also includes first, second and third one-way valves 63a, 63b and 63c.

In the embodiment shown in FIG. 6, a first conduit 64 is fluidly coupled between the first tubular port 62a and first one-way valve 63a. A second conduit 65 is coupled between the second tubular port 62b and second one-way valve 63b. A third conduit 66 is coupled between the third tubular port 62c and a third one-way valve 63c. A fourth conduit 67 is coupled to the outlets of the first, second and third one-way valves 63a, 63b and 63c. First and second one-way valves 63a, 63b are configured to allow a one-way flow of fluid from fourth conduit 67 into the tubular ports 62a, 62b as shown by arrow A. Third one-way valve 63c is configured to allow one-way flow of fluid from fluid container 35 and tubular port 62b as shown by arrow B to fourth conduit 67. The conduits 64 and 65 and one-way valves 62a and 62b are of sufficiently large caliber and length to allow free and constant flow of fluid and/or air into aspiration container 37 as inner cartridge 24 is displaced from its first position to second position thereby retracting aspiration plungers 59 in the proximal direction. The conduit 66 and one-way valve 62c are of sufficiently large caliber and length to allow for a relatively constant volumetric flow of fluid from fluid container 35 into fourth conduit 67 as the energy storage member 31 is displaced from its first to second configuration to provide a force on the plunger assembly 33 and advance the plunger assembly 33 from its first position to second position.

To illustrate operation of the delivery device 20, a sequence of events illustrating the aspiration and delivery of fluid by the device, as well as the position of the various components, is discussed progressing from FIGS. 7A-7B through FIGS. 9A-9B.

FIGS. 7A and 7B illustrate the delivery device 20 in an initial position, where the delivery device 20 is ready for use and activation. Referring to FIGS. 7A and 7B, fluid container 35 is disposed within inner cartridge 24 and aspiration container 37 is disposed within outer cartridge 22. Spring 31 is shown compressed in a first configuration between the proximal end of inner housing 56 and the proximal end of plunger 33b. The actuator 26 is initially at the distal end of the opening 23c. The plunger assembly 33 is held in place by the releasable retainer member 34, the releasable retainer member 34 being in tension and lodged between the plunger assembly 33 and the cutout section 40 of the distal end of inner cylindrical wall 42. Actuator 26, inner cartridge 24, spring 31, plunger assembly 33, fluid container 35, releasable retainer member 34 and aspiration plunger 59 are all operatively coupled to one another.

In the first configuration shown in FIGS. 8A-8B, actuator 26 is activated by sliding it towards the proximal end of opening 23c. Inner cartridge 24 is therefore moved within outer cartridge 22 in a proximal direction A. Correspondingly, aspiration plungers 59 are also moved within aspiration containers 37 in the proximal direction A thereby drawing fluid and/or air from third conduit 67 into the aspiration containers 37. One-way valves 63a and 63b prevent the backflow of fluid and/or air. The latch tabs 57 and the shaped proximal end of plunger rod 33a of the plunger assembly 33 protrude into the inner cross-sectional area formed by inner cylindrical wall 42 of outer cartridge 22 thereby biasing the latch tabs 57 in an outward position to release the plunger assembly 33. A force produced by the spring 31 acts on the plunger assembly 33 to begin movement of the plunger assembly 33 in a distal direction B to begin dispensing fluid contained in the fluid container 35 through third conduit 66 and fourth conduit 67. Engagement between latch tabs 57 and the distal end of inner cylindrical wall 42 prevents the inner cartridge 24 from moving in the direction B.

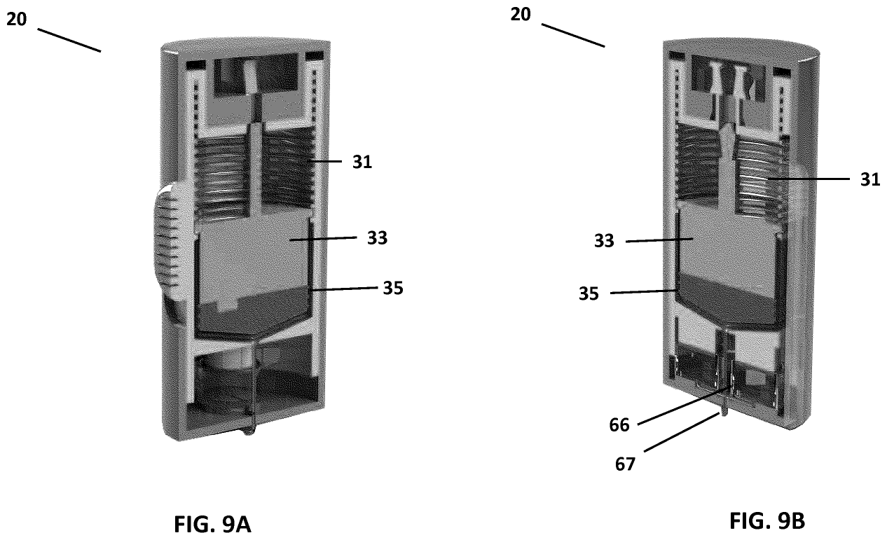

In the second configuration shown in FIGS. 9A-9B, the spring 31 is fully extended and has displaced the plunger assembly 33 to the distal end of the fluid container 35. The fluid contained within the fluid container 35 has been substantially delivered through the third and fourth conduit 66, 67 respectively.

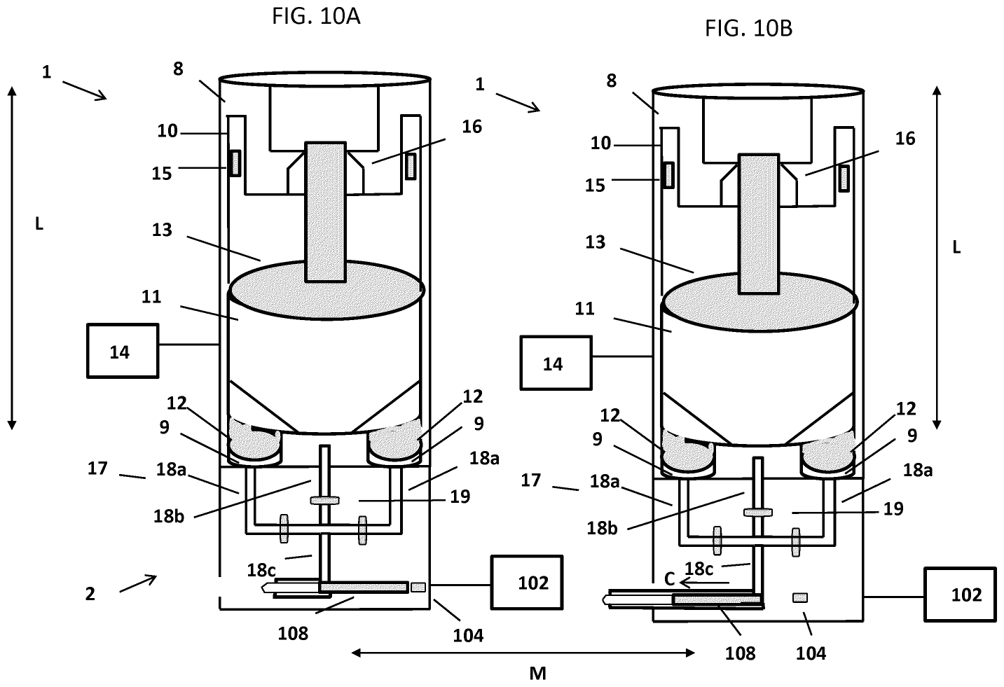

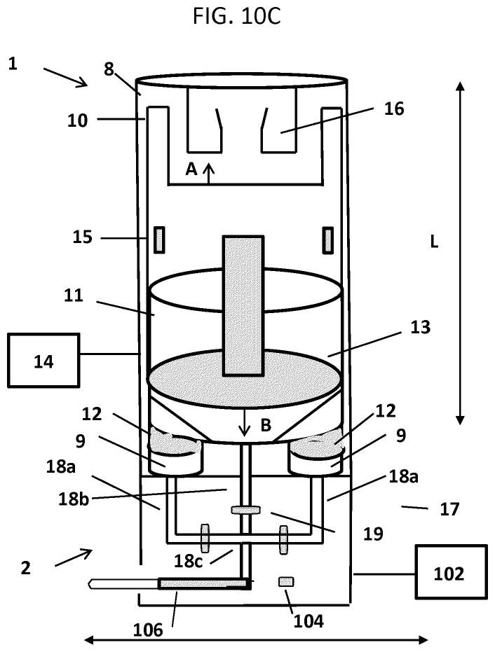

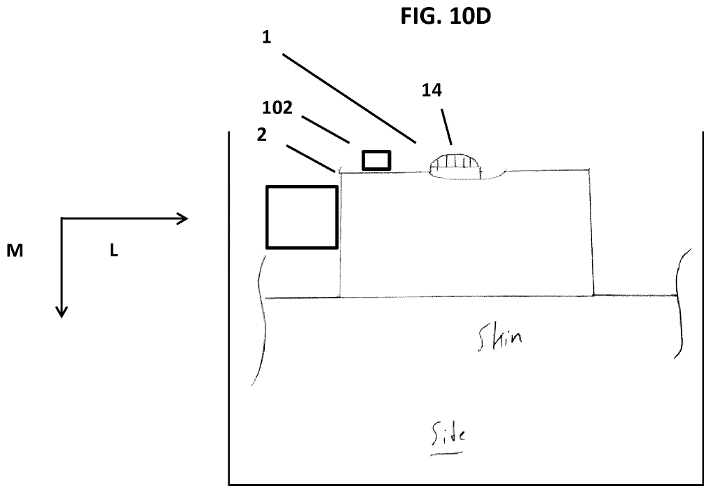

Turning now to FIGS. 10A and 10B, a cross-sectional view of the delivery device 1 depicted in FIG. 1A and described above is shown according to another embodiment where the delivery device 1 further includes an IO access device 2. In particular, FIG. 10A depicts the IO access device 2 in a first configuration and FIG. 10B depicts the IO access device 2 in a second configuration. The remaining components of the delivery device 1 in FIGS. 10A and 10B are depicted in their initial positions as shown in FIG. 1A and described above.

The IO access device 2 is configured for accessing the IO space of a subject and is coupled to the third fluid conduit 18c of the fluid delivery system 17. The IO access device 2 generally includes a tissue penetrator assembly 108 configured to penetrate a bone of the subject, a driver 104 operably coupled to the tissue penetrator assembly 108 to drive at least a portion of the tissue penetrator assembly 108 into the IO space of a subject and a second actuator 102 operable to activate the driver 104. In some embodiments, the tissue penetrator 108 may include a needle disposed within a flexible catheter such that the needle may be removed once the tissue penetrator assembly 108 has penetrated the IO space.

The IO access device 2 can generally be operated by positioning the IO access device 2 at a suitable location over a bone of a subject into which it is desired to inject a fluid. For example, the bone may be a sternum, tibia, humerus or other intraosseous injection site. Second actuator 102 may then be activated to drive tissue penetrator assembly 108 in a distal direction. The second actuator 102 may be any suitable device, such as, for example, a handle, a lever, a push button, a slidable button or a trigger. Upon activation of the second activator 102, the driver 104 is activated and the tissue penetrator assembly 108 is displaced within the outer cartridge 8, as indicated by arrow C in FIG. 10B, along an axis M perpendicular to longitudinal axis L from a first position (FIG. 10A) to a second position (FIG. 10B) by a force produced by the driver 104. As illustrated, when the tissue penetrator assembly 108 is displaced in a distal direction from the first position to second position, the tissue penetrator assembly 108 (along with third fluid conduit 18c) is configured to access the IO space of the subject while the delivery device 1 is still in the initial position as shown in FIG. 1A.

The driver 104 provides power to the tissue penetrator assembly 108. The power is sufficient to penetrate the skin, muscle and bone of the subject and may be supplied to the tissue penetrator assembly 108 by any suitable means, such as, one or more of the following: a battery, a spring, compressed gas, manual force, and any other mechanical or electrical source of rotation or reciprocation. The power may also be supplied directly or indirectly (e.g. using gears) by the operator and/or the subject. In addition to batteries, electric power may come from any other suitable source including conventional wall outlets. The power source may be operably coupled with a motor. Motors may be selected from the group consisting of DC motors, AC motors, compressed gas motors, wound spring motors, and reciprocating motors. Motors may also be coupled to one or more gears, which may optionally be positioned in one or more gear boxes.

Once the IO access device 2 has moved from its first configuration to its second configuration, the delivery device 1 may be operated in a substantially similar manner as shown in FIGS. 1A and 1B and as described above. In particular, with reference to FIG. 10C, the operator can activate the first actuator 14 to cause a sequence of movements whereby the inner cartridge 10, along with aspiration plunger 12, are displaced within the outer cartridge 8 in a proximal direction (arrow A) to aspirate third fluid conduit 18c and the plunger assembly 13 is subsequently displaced in a distal direction (arrow B) within the inner container 11 to force the fluid confined therein through the second and third fluid conduits 18b and 18c and to the subject at a relatively constant volumetric flow rate. The embodiment shown in FIG. 10D depicts the IO access device 2 and delivery device 1 positioned on the skin of the subject prior to use.

Although shown as a unitary device, in some embodiments, the IO access device 2 may be decoupled from the delivery device 1 after the IO access device has moved to its second configuration (i.e. after the tissue penetrator assembly 108 has penetrated the IO space). In such embodiments, the IO access device 2 may further include a removable base support (not shown) positioned at the distal end of the tissue penetrate assembly 108 and the fluid delivery system 17 may include a wye/tee connection (not shown). Thus, once the IO access device 2 has been decoupled from the delivery device 1, the base support will be positioned near the skin and is configured to hold the fluid delivery system 17 secure with one port of the tee connection in fluid communication with the fluid conduit 18c and a second port open.

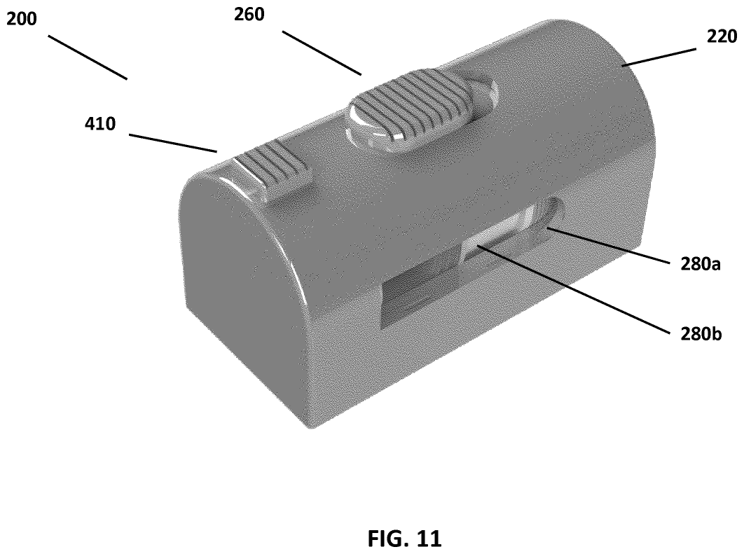

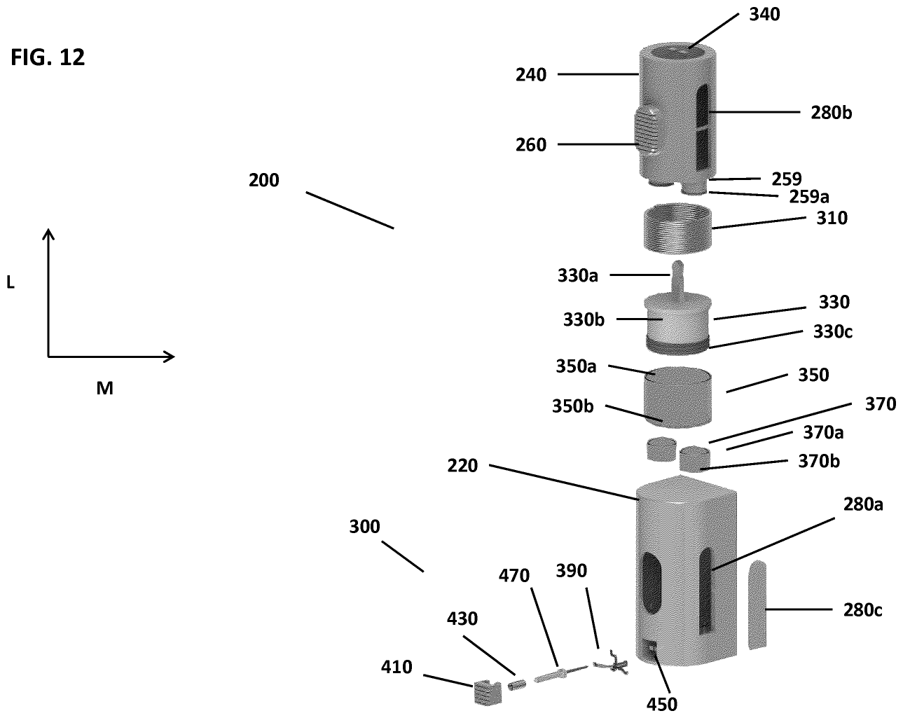

With reference now to FIGS. 11 and 12, a delivery device according to a second embodiment is shown and generally designated by reference numeral 200, the delivery device 200 further comprising an IO access device 300 disposed therein. As shown in FIGS. 11 and 12, the delivery device 200 is generally similar to the delivery device 20 described above and includes an outer cartridge 220 with a coupling end 450 disposed therein, an inner cartridge 240 configured to be movably disposed within the outer cartridge 220, a first actuator 260, first and second elongated windows 280a, 280b configured to allow an operator to view the contents within the delivery device 200, an energy storage member 310, a plunger assembly 330, a releasable retainer member 340, a fluid container 350, an aspiration container 370, a fluid delivery system 390 while the IO access device 300 generally includes a second actuator 410 configured to be releasably coupled to the coupling end 450, a driver 430 and a tissue penetrator 470 configured to be releasably coupled to coupling end 450. In some embodiments, the windows 280a, 280b may be open, with no material or they may comprise a clear material 280c, such as a translucent or transparent material, to allow the operator to view the contents of the fluid container 350 within the delivery device 200.

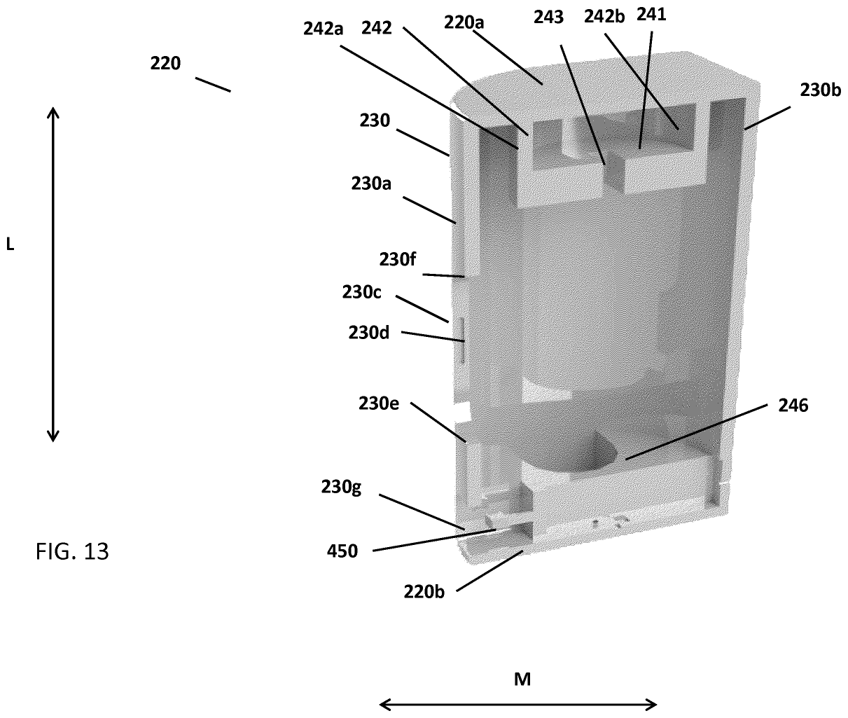

With reference now to FIGS. 12 and 13, the outer cartridge 220 like outer cartridge 22 described above, is generally shaped and dimensioned to fit within an operator's hand and includes a wall 230 with an exterior surface 230a and an interior surface 230b. While a unitary (i.e., one-piece) wall 230 has been illustrated in FIG. 13 that defines both the exterior and interior surfaces 230a, 230b, it will be understood that according to other embodiments the wall 230 may include a plurality of layers with different layers defining the exterior and interior surfaces 230a, 230b. Wall 230 may further include a first opening 230c to accommodate the first actuator 260 of inner cartridge 240 which is operated by the operator. Opening 230c is configured to allow first actuator 260 to be movably displaced in the longitudinal direction L due to the presence of two opposing rails 230d positioned on the walls of the first opening 230c. The rails 230d lead into an internal distal stop end 230e and an internal proximal stop end 230f limiting the distance the first actuator 260 can travel in the L direction within the first opening 230c. In some embodiments, the rails 230d may have a length of between about 5 mm to about 10 mm. Wall 230 further includes a second opening 230g to accommodate the second actuator 410 which is operated by the operator. Second opening 230g is configured to allow the second actuator 410 to be movably displaced in a direction M perpendicular to longitudinal direction L due to the presence of two opposing rails (not shown) positioned on the walls of the second opening 230g. Coupling end 450 limits the distance the second actuator 410 can travel within the second opening 230g. Wall 230 further includes a third opening (not shown) positioned on the opposing side from opening 230g (in some embodiments including a septum which seals opening 203g) through which the tissue penetrator assembly 470 and third fluid conduit 218c can be extended (or which can pierce septum) when the driver 430 is activated as further described below.

According to certain embodiments of the present disclosure, the wall 230 may be rigid. According to other embodiments, the wall 230 may be flexible, whether according to the nature of the material that defines the wall 230 or according to the nature of the structure of the wall 230. The wall 230 may be made of glass, metal, or polymer, for example. In particular, polymer versions may be made of polycarbonate, polypropylene, polyethylene (such as high density polyethylene), polytetrafluoroethylene, cyclic olefin polymer, cyclic olefin copolymer, crystal zenith olefinic polymer, nylon, or engineering resins. As to flexible versions of the wall 230, butyl rubber, silicon-based rubber, latex-based rubber, coated rubber, as well as multi-layer polymer films, such as polyethylene (such as low density polyethylene) and polypropylene, may be used.

The wall 230 may have a generally cylindrical shape (although other shapes are contemplated, such as rectangular). According to some embodiments, the wall 230 may have a length of between about 70 mm to about 140 mm and defines two opposed, closed ends, proximal end 220a and distal end 220b, having a diameter of between about 20 mm to about 50 mm. Attached to the interior surface of proximal end 220a is an inner cylindrical wall 242 having an exterior surface 242a and an interior surface 242b. Inner cylindrical wall 242 may be rigid or flexible and in some embodiments may have a length of between 10 mm to about 30 mm, defines an inner cylindrical cross-section having a closed distal end 241 comprising a cutout section 243. The closed distal end 241 therefore has a diameter that is less than the diameter of proximal end 220a and distal end 220b. In some embodiments, the diameter of the closed distal end 241 may be about 20 mm to about 40 mm. The cutout section 243 is shaped and configured to engage the retainer member 340 (not shown) when the inner cartridge 240 is in its first position. For instance, the wall the cutout section 243 may be sized and adapted to correspondingly mate with the shape of the retainer member 340 when the inner cartridge 240 is in the first position and to allow plunger assembly 330 but be movably disposed therein when the inner cartridge is moved to its second position as further described below.

The exterior surface 242a of inner cylindrical wall 242 and the interior surface 230b of wall 230 are spaced apart a sufficient distance to allow the inner cartridge 240 to be movably disposed therein in a longitudinal direction L when the first actuator 260 is activated.

An aspiration container mount 246 is attached to the interior surface of the distal end 220b and may be formed of the same material as the wall 230 or a different rigid solid material, such as plastic. The aspiration container mount 246 is sized and adapted to hold the aspiration container 237 (not shown) in alignment with an aspiration plunger 259 (not shown) of inner cartridge 240.

The delivery device 200 is configured to include an aspiration container 370 disposed within the outer cartridge 220. The aspiration container 370 is positioned on or held by the aspiration mount 246. In some embodiments, the aspiration container 370 may have a length of about 4 mm to about 12 mm. The aspiration container 370 includes an open proximal end 370a and a closed distal end 370b with a diameter of between about 12 mm to about 16 mm when two aspiration containers are present or about 20 mm to about 50 mm when one aspiration container is present. The proximal end of the aspiration container 370 is sized and adapted to receive the aspiration plunger 259 (not shown) of inner cartridge 240. The closed distal end 370b includes an orifice fluidly coupled to the fluid delivery system 390, such as by a luer connector, threads, a snap-fit, a latch, a lock, a friction fit coupling, or any other suitable coupling features. The orifice may have a diameter of between about 0.159 mm to about 1.6 mm. In some embodiments, the aspiration container 370 can be, for example, a glass vial.

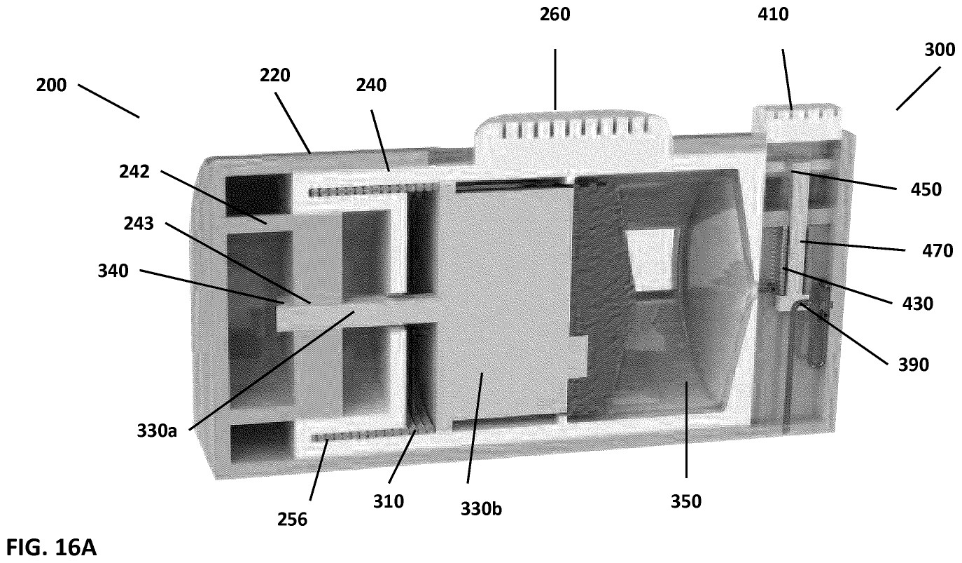

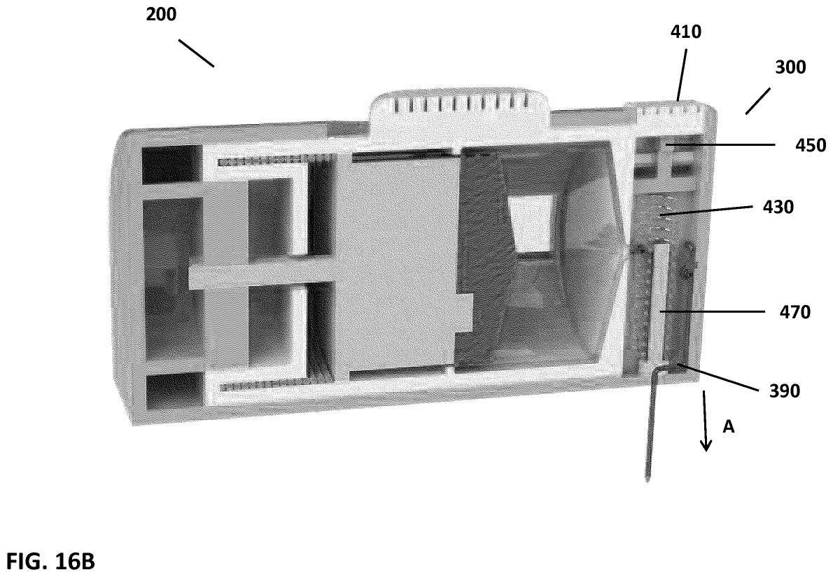

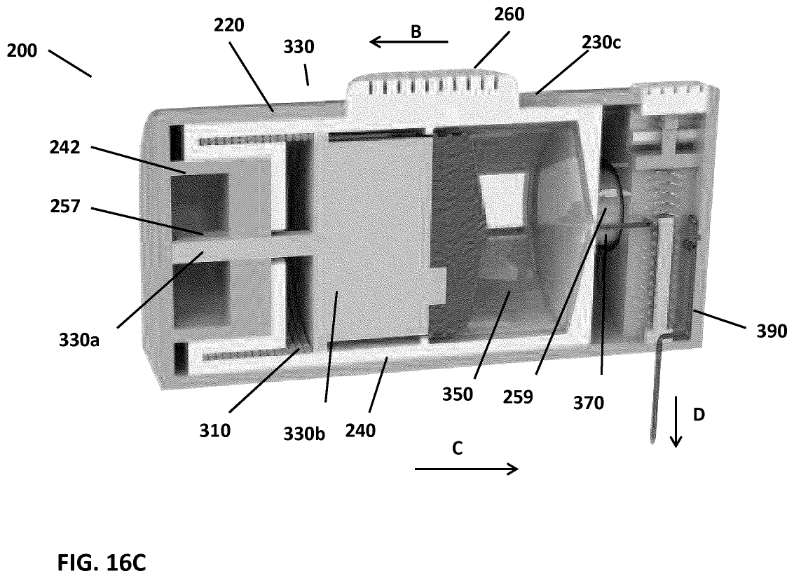

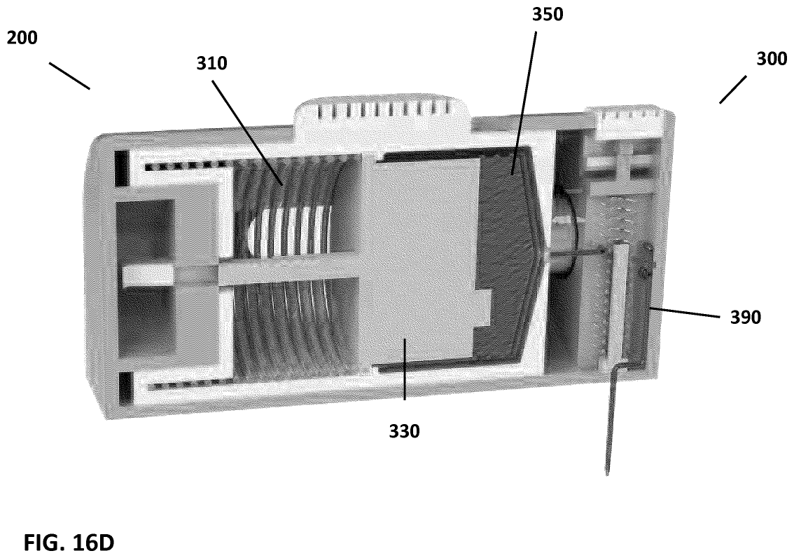

Outer cartridge 220 further includes a coupling end 450 positioned at its distal end 220b and configured to secure tissue penetrator assembly 470 in its first position during storage and to release tissue penetrator 470 upon activation of the second actuator 410 as further described below. The coupling end 450 can be any suitable mechanism for securing and deploying the tissue penetrator assembly 470, such as, for example, a mechanical linkage, a spring-loaded rod, a tensioned latch or tab or the like. In the embodiment shown in FIG. 12, coupling end 450 is in the form of one or more latch tabs inherently biased outwards and configured to be in inter engagement with the distal end of the second actuator 410 when the second actuator 410 and the tissue penetrator assembly 470 are in their initial positions (see FIG. 16A).

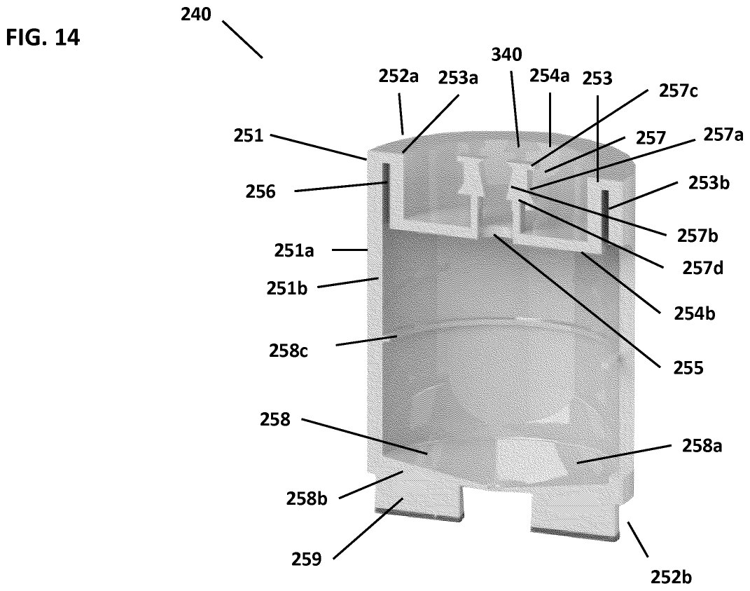

With reference now to FIGS. 12 and 14, inner cartridge 240 includes a wall 251 with an exterior surface 251a and an interior surface 251b. Similar to outer cartridge 220, while a unitary (i.e., one-piece) wall 251 has been illustrated in FIG. 14 that defines both the exterior and interior surfaces 251a, 251b, it will be understood that according to other embodiments the wall 251 may include a plurality of layers with different layers defining the exterior and interior surfaces 251a, 251b. Accordingly, wall 251 may be made of glass, metal, or polymer, for example. In particular, polymer versions may be made of polycarbonate, polypropylene, polyethylene (such as high density polyethylene), polytetrafluoroethylene, cyclic olefin polymer, cyclic olefin copolymer, crystal zenith olefinic polymer, nylon, or engineering resins.

Wall 251 is sized and configured to be movably disposed within outer cartridge 220 and thus may have a correspondingly similar, but smaller, shape as the outer cartridge 220, such as a generally cylindrical (or rectangular) shape. According to some embodiments, wall 251 may have a length of about 60 mm to about 140 mm and may define two opposed ends, a semi-enclosed proximal end 252a and an open distal end 252b. The semi-enclosed proximal end 252a includes an inner cylindrical wall 253, which may be rigid or flexible, attached to its interior surface. Inner cylindrical wall 253, which in some embodiments may a length of between 25 mm to about 45 mm, includes an interior surface 253a and an exterior surface 253b and defines two opposed ends, an open proximal end 254a and a closed distal end 254b comprising a cutout section 255. The cutout section 255 is shaped and configured to prevent the plunger rod 330a (not shown) of the plunger assembly 330 from being movably disposed within inner cartridge 240 when the inner cartridge 240 is in its first position and be movably disposed in the longitudinal direction L after the first actuator 260 has been activated. For instance, the wall of the cutout section 255 may be sized and adapted to correspondingly mate with the shape of the plunger assembly 330 when the inner cartridge 240 is in its first position and to allow plunger assembly 330 to be movably disposed therein when the inner cartridge 240 is moved to its second position as further described below.

The exterior surface 253b of inner cylindrical wall 253 and the interior surface 251b of wall 251 are spaced apart to form an inner housing 256. Inner housing 256 is sized and adapted to allow the energy storage member 310 (not shown) to be movably disposed therein from its first configuration to second configuration after activation.

First actuator 260, in the form a slide button, is attached to the exterior surface 251a of wall 251. The top surface of first actuator 260 is ribbed in a direction perpendicular to the direction of actuator movement, as shown, to allow the operator's finger to engage the actuator 260 without slipping. The first actuator 260 is initially in the position shown in FIG. 16A and is advanced in the proximal direction (i.e., in the direction towards the proximal end 220b of the outer cartridge 220) to its second position shown in FIG. 16C by the operator during operation of the device 200. As will be described in detail below, movement of the first actuator 260 from its first position to second position causes a sequence of movements of the components within the delivery device that subsequently lead to the delivery of fluid contained within the delivery device.

The releasable retainer member 340, shown in the form of one or more latch tabs 257, is fixedly attached to the interior surface of distal end 254b of inner cylindrical wall 253. The latch tabs 257 have a downwardly facing outward shoulder 257a, a downwardly facing inward shoulder 257b, an upper flange 257 and a lower flange 257d. The latch tabs 257 are inherently biased outwardly, with the wall of the cutout section 243 of outer cartridge 220 being shaped and configured so as to interferingly engage downwardly facing outward shoulder 257a under force of movement of the energy storage member 310 when the delivery device 200 is in an initial position. In addition, downwardly facing inward shoulder 257b and lower flange 257c are shaped and configured to interferingly engage the plunger rod 330a (not shown) of the plunger assembly 330 when the delivery device 200 is in the position. With this arrangement, movement of the plunger assembly 330 in the L direction is limited due to inter engagement between the downwardly facing outward shoulder 257a of the latch tabs 57 and the cutout section 243 of outer cartridge 220 as well as by the inter engagement between downwardly facing inward shoulder 257b and lower flange 257d of the latch tabs 57 and the plunger rod 330a of plunger assembly 330. The plunger assembly 330 is thus retained before use. Once the inner cartridge 240 has moved in a proximal direction from its first position to second position (i.e. upon activation of the first actuator 260), latch tabs 257 will also have moved to a position within the inner cylindrical cross-section defined by inner cylindrical wall 242 of outer cartridge 220 thus allowing the latch tabs 257 to spring outwards and thereby releasing the plunger assembly 330 from engagement. Additionally, once the latch tabs 257 have moved to the position within the inner cylindrical cross-section defined by inner wall 242, lower flange 57d of the latch tabs 57 will now be in inter engagement with the upper surface of distal end 241 of the inner cylindrical wall 242 thereby preventing the inner cartridge 240 from being displaced in the distal direction (see FIG. 16D).

The inner cartridge 240 also includes a fluid container mount 258 sized and adapted to hold fluid container 350. The fluid container mount 258 includes an upper surface 258a for engaging the distal end of the fluid container 350 and a lower surface 258b and an inner ring portion 258c. An aspiration plunger 259 is movably coupled to the lower surface 258b. The aspiration plunger 259 is sized and adapted to be movably disposed within the aspiration container 370 when the inner cartridge 240 is moved from its first position to second position. The sidewalls of the aspiration plunger 259 can be configured to contact the interior surfaces of the sidewalls of the aspiration container 370 such that the aspiration plunger 259 forms a fluid-tight seal with the sidewalls of the aspiration container 370. In some embodiments, the aspiration plunger 259 may include a plug 259a positioned at its distal end to engage the inner sidewalls of the aspiration container 370.

The delivery device 200 is therefore configured to include a fluid container 350 disposed within the inner cartridge 240 mounted on the fluid container mount 258. The fluid container 350 contains (i.e., is filled or partially filled with) a fluid. The fluid container 350 includes a proximal end 350a and a distal end 350b that is fluidly coupled to the fluid conduit 366 of fluid delivery system 390 (not shown), such as by a luer connector, threads, a snap-fit, a latch, a lock, a friction fit coupling, or any other suitable coupling features. The fluid container 350 may contain a volume of about 1 ml to about 20 mL and have a length of between about 10 mm to about 40 mm. The proximal end 350a may have a diameter of between about 25 mm to about 40 mm and the distal end 350b may have a diameter of between about 2 mm to about 3 mm. The orifice of the distal end may have a diameter of between about 0.05 mm to about 1.6 mm.

In the embodiment shown in FIG. 12, the energy storage member 310 of delivery device 200 is a mechanical energy storage member comprising a spring, such as, for example, a helical, compression, extension, torsion, constant, variable, variable stiffness or any other type of spring having a spring constant ranging between about 1 N/m to about 500 N/m. The energy storage member 310 is operatively coupled to the plunger assembly 330 and produces a force on the plunger assembly 330 when the energy storage member 310 moves from its first configuration to second configuration after the first actuator 260 has been activated.

As shown in FIG. 12, the plunger assembly 330 comprises a plunger rod 330a and plunger 330b. In some embodiments, the plunger assembly 330 may be formed as a single piece (as shown) or modular components in other embodiments. The modular components may be fixed to one another or located adjacently, but not connected, so as to move together.

A distal end portion of the plunger assembly 330 is configured to be disposed within fluid container 350. The distal end portion of the plunger assembly 330 can be coupled to and/or in contact with a plug 330c which is configured to be in fluid communication with the fluid disposed within an internal volume defined by the fluid container 350. The distal end portion of the plunger assembly 330 is configured to be movably displaced within the internal volume defined by the fluid container 350 due to the force produced by the energy storage member 310 when it moves from its first configuration to second configuration. In this manner, the energy storage member 310 acting on the plunger assembly 330 can displace the plug 330c within the fluid container 350 to expel the fluid through the orifice of distal end 350b of the fluid container 350. The sidewalls of the plug 330c can be configured to contact the interior surfaces of the sidewalls of the fluid container 350 such that the plug 330c forms a fluid-tight seal with the sidewalls of the fluid container 350, for example, to prevent leakage of the fluid. The plug 330c can be made of an inert and/or biocompatible material which is rigid but soft. Example materials include rubber, silicone, plastic, polymers, any other suitable material or combination thereof. In some embodiments, the plug 330c can be monolithically formed with the plunger assembly 330.

The plunger rod 330a is attached to the proximal end of plunger 330b and includes an elongated lower member and a shaped upper member sized and configured to engage with the downwardly facing inward shoulder 257b and lower surface of upper flange 257c of the latch tabs 257 when the plunger assembly 330 is in its first position. In the embodiment shown in FIG. 12, the shaped upper member of the plunger rod 330a has an upwardly pointing arrow-shape, however any shape may be utilized so long as it engages with downwardly facing inward shoulder 257b and lower surface of upper flange 257c of the latch tabs 257. Plunger 330b is generally cylindrically shaped having a proximal end and a distal end and is configured to be movably disposed within the interior volume defined by the fluid container 350.

As described above, the fluid container 350 defines an internal volume configured to house a fluid. The fluid may comprise a medicament such as, but not limited to those fluids described above.

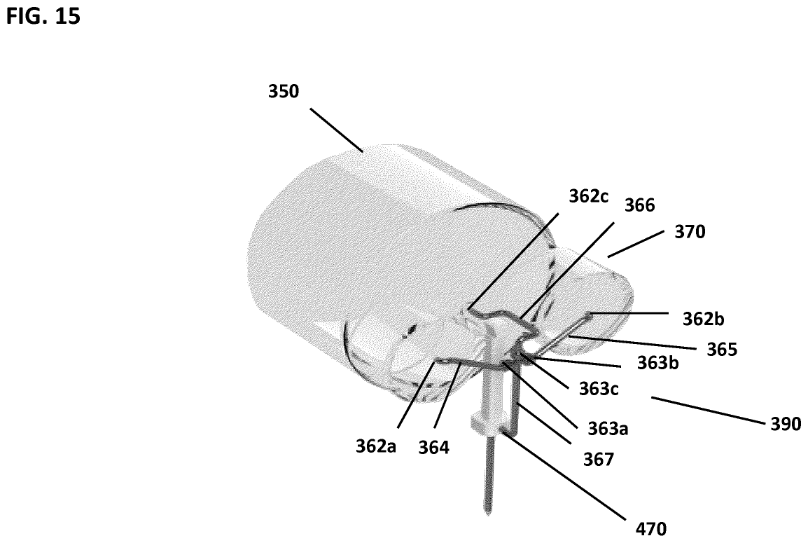

With reference now to FIGS. 12 and 15, delivery device 200 includes fluid delivery system 390 configured to fluidly couple to the orifice of the distal end 350b of fluid container 350 and to the orifice of the distal end 370b of aspiration container 370. In the FIG. 15 embodiment, the fluid delivery system 390 includes first, second and third tubular ports 362a, 362b and 362c. First and second tubular ports 362a, 362b are configured to fluidly couple with the orifices of the aspiration containers 370. Third tubular port 362c is configured to fluidly couple with the orifice of fluid container 350. Fluid delivery system 390 also includes first, second and third one-way valves 363a, 363b and 363c.

In the embodiment shown in FIG. 15, a first conduit 364 is fluidly coupled between the first tubular port 362a and first one-way valve 363a. A second conduit 365 is coupled between the second tubular port 362b and second one-way valve 363b. A third conduit 366 is coupled between the third tubular port 362c and a third valve 363c. A fourth conduit 367 is coupled to the outlets of the first, second and third one-way valves 363a, 363b and 363c. First and second one-way valves 363a, 363b are configured to allow one-way flow of fluid from fourth conduit 367 into the tubular ports 362a, 362b and into aspiration container 370. Third one-way valve 363c is configured to allow one-way flow of fluid from fluid container 350 and tubular port 362b to fourth conduit 367 and to tissue penetrator 470. The first and second conduits 364 and 365 and one-way valves 362a and 362b are of sufficiently large caliber and length to allow free and constant flow of fluid and/or air into aspiration container 370 as inner cartridge 240 is displaced from its first position to second position acts thereby retracting aspiration plungers 259 in the proximal direction. The third conduit 366 and one-way valve 362c are of sufficiently large caliber and length to allow for a relatively constant volumetric flow of fluid from fluid container 350 into fourth conduit 367 as the energy storage member 310 is displaced from its first to second configuration to provide a force on the plunger assembly 330 and advance the plunger assembly 330 from its first position to second position.

With reference again to FIGS. 12 and 15, the delivery device 200 further includes an IO access device 300. The IO access device 300, coupled to the fourth conduit 367 of the fluid delivery system 390 such as by a luer connector, threads, a snap-fit, a latch, a lock, a friction fit coupling, or any other suitable coupling features, is configured for accessing the IO space of a subject and delivering fluid from the fluid delivery system 390 to the IO space. Although shown as forming a unitary device, the IO access device 300 may be configured such that it can be decoupled from the delivery device 200.

The IO access device 300 includes a second actuator 410 in the form of a push button. The top surface of the second actuator 410 is ribbed in a direction perpendicular to the direction of actuator movement, as shown, to allow the operator's finger to engage the second actuator 410 without slipping. The second actuator 410, configured to be directly or indirectly coupled to the coupling end of outer cartridge 220, can be activated by applying a force to the top surface of the second actuator 410 in the M direction to advance the second actuator 410 from its initial (or first position) to a second position. Movement by the second actuator 410 from its first position to second position causes a sequence of movements of the components of the IO access device 300 which move the IO access device 300 from a first configuration to a second configuration as further described below.

The IO access device 300 also includes a driver 430. According to the embodiment shown in FIG. 12, driver 430 includes a spring operably coupled to the tissue penetrator assembly 470. The spring may be directly or indirectly coupled to the coupling end 450 of outer cartridge 220 fixing the position of that end of the spring. Upon movement of the second actuator 410 from its first position to second position, the coupling end 450 of outer cartridge 220 and the tissue penetrator assembly 470 are released from inter engagement with the second actuator 410 allowing the latch tabs of the coupling end 450 to spring outwards thereby releasing the tissue penetrator assembly 470 from engagement with the coupling end 450. The driver 430 provides the power to drive at least a portion of the tissue penetrator assembly 470 into the IO space of the subject.

The tissue penetrator assembly 470 is configured to be releasably coupled to coupling end 450 of outer cartridge 220 at its proximal end and may include in various combinations a shaft, a needle, a needle set, a cannula, a trocar, a stylet or a catheter. Needles suitable for the present disclosure may be from about 30 gauge to about 10 gauge and may be sized to provide a penetration depth of between about 15 mm to about 45 mm. The tissue penetrator assembly 470 is further configured to couple to the third conduit 367 of the fluid delivery system 390 at its distal end such as described above.

To illustrate operation of the delivery device 200, a sequence of events illustrating accessing the IO space by the IO access device 300 and the aspiration and delivery of fluid by the delivery device, as well as the position of the various components, is discussed progressing from FIGS. 16A-16D.

FIG. 16A illustrates the delivery device 200 and IO access device 300 in their initial positions. The tissue penetrator assembly 470 is held in place by coupling end 450 of outer cartridge 220 and second actuator 410. Driver 430 is shown compressed between the tissue penetrator assembly 470 and lower surface of coupling end 450. Second actuator 410, tissue penetrator assembly 470 and driver 430 are operatively coupled and in their first positions. Fluid delivery system 390 is coupled to the tissue penetrator assembly 470 and fluid container 350, fluid container 350 being disposed within inner cartridge 240. Aspiration container 370 (see FIG. 16C) is disposed within outer cartridge 220. Spring 310 is shown compressed in a first configuration between the proximal end of inner housing 256 and the proximal end of plunger 330b. The first actuator 260 is initially at the distal end of the opening 230c. The plunger assembly 330 is held in place by the retainer member 340, the retainer member 340 being in tension and lodged between the plunger rod 330a and the cutout section 243 of inner cylindrical wall 242. Inner cartridge 240, spring 310, plunger assembly 330, retainer member 340 and aspiration plunger 259 are all operatively coupled to one another and will move as one when the first actuator 260 is activated.

With reference to FIG. 16B, the operator can activate the IO access device 300 to move the IO access device from a first configuration to second configuration by pushing down on the second actuator 410 to move second actuator 410 from its first position to second position thereby releasing tissue penetrator assembly 470 from engagement with coupling end 450. Once tissue penetrator assembly 470 has been released, driver 430 drives tissue penetrator assembly 470 (along with the third conduit of fluid delivery system 390) towards the subject and into the IO space of the subject as shown by arrow A. Third conduit of fluid delivery system 390 has just enough slack to uncoil and become taut once the tissue penetrator assembly 470 has accessed the IO space. The IO access device 200 remains in its initial position as shown in FIG. 16A.

With reference to FIG. 16C, the delivery device 200 may then be moved from its initial position to a first configuration by activating the first actuator 260. Actuator 260 is activated by sliding it towards the proximal end of opening 230c. Inner cartridge 240 is therefore moved within outer cartridge 220 in a proximal direction B. Correspondingly, aspiration plunger 259 is also moved within aspiration container 370 in the proximal direction B thereby drawing fluid and/or air from the fluid delivery system 390 into the aspiration container 370. One-way valves (not shown) of fluid delivery system 390 prevent the backflow of fluid and/or air. The latch tabs 257 and the shaped proximal end of plunger rod 330a protrude into the inner cross-sectional area formed by inner cylindrical wall 242 of outer cartridge 220 thereby biasing the latch tabs 257 in an outward position to release the plunger rod 330a. A force produced by the spring 310 acts on the plunger 330b to begin movement of the plunger assembly 330 in a distal direction C to begin dispensing fluid contained in the fluid container 350 through the fluid delivery system 390 as shown by arrow D.

In the delivery device's second configuration shown in FIG. 16D, the spring 310 is fully extended and has displaced the plunger assembly 330 to the distal end of the fluid container 350. The fluid contained within the fluid container 350 has been substantially delivered through the fluid delivery system 390 to the subject.

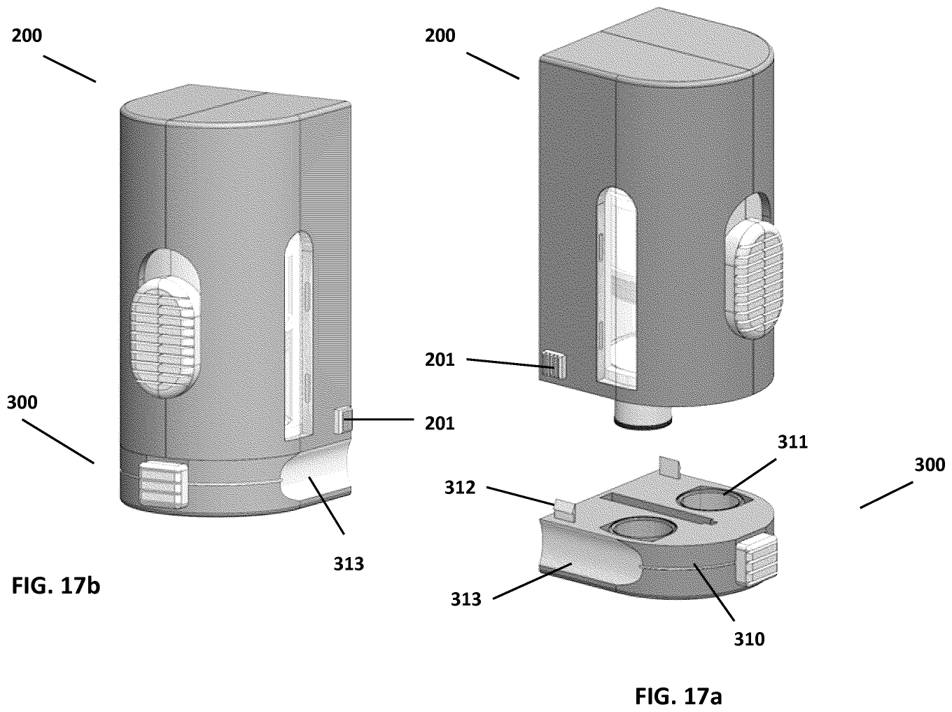

With reference now to FIG. 17A, an embodiment is illustrated where the IO access device 300 is separate from the delivery device 200 as seen prior to connecting for use. In this embodiment, the components and operation of the delivery device 200 and IO access device 300 are substantially similar to that for the delivery device 200 and IO access device 300 described above with the following changes. In the embodiment shown in FIG. 17A, the delivery device 200 includes a catch mechanism 201 comprising an inner indentation (not shown) configured to couple to an engaging lock 312 of the IO access device 300 to secure the IO access device to the delivery device as further described below. In addition, the coupling end 450 of the outer cartridge has been moved to the proximal end of the IO access device 300 (not shown) for releasably coupling to the tissue penetrator assembly 470. In addition, the IO access device 300 is now disposed within housing 310. Housing 310 includes an aspiration container mount 311 configured to hold an aspiration container (not shown). Housing 310 also includes side cut-out sections 313 on opposing sides to allow the operator to properly align the delivery device 200 and IO access device 300 for use.

The IO access device 300 and the delivery device 200 may be connected by pressing the catch mechanism 201 to expose inner indentation, aligning the IO access device 300 and delivery device 200 and releasing the catch mechanism 201 to securely couple the engaging lock 312 and inner indentation (see FIG. 17B). The delivery device 200 may be released from the IO access device by pressing the catch mechanism 201 to decouple the engaging lock 312 from the inner indentation.

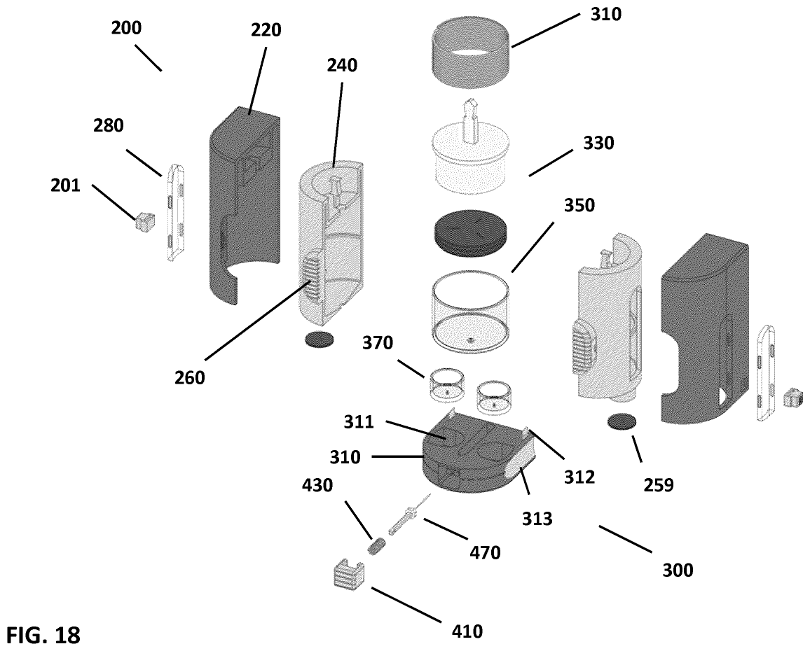

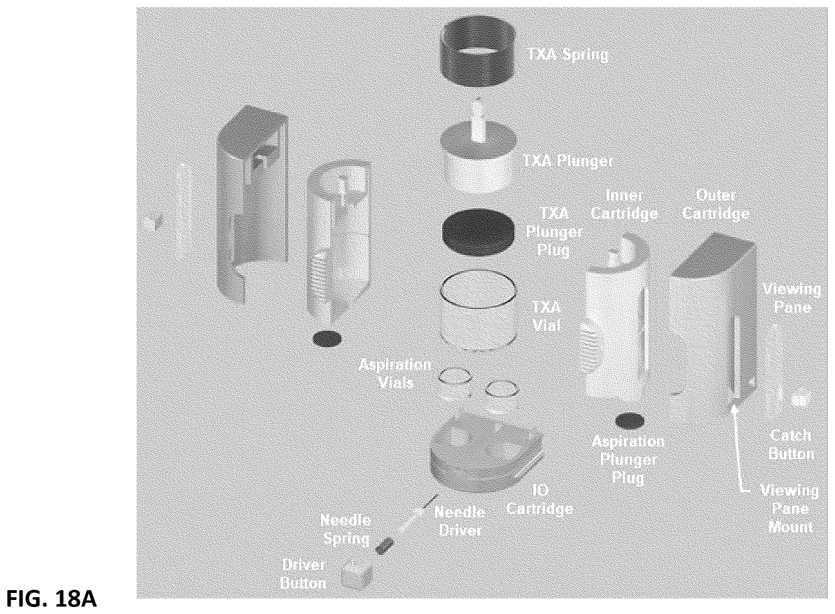

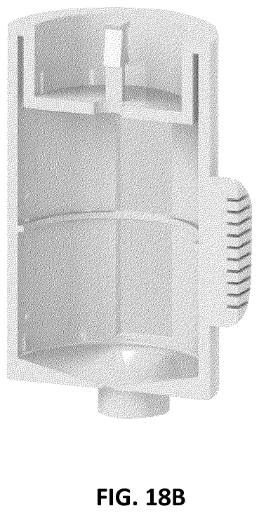

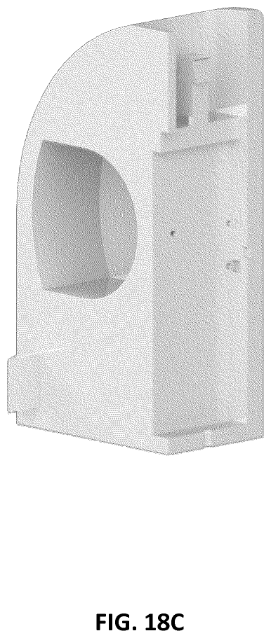

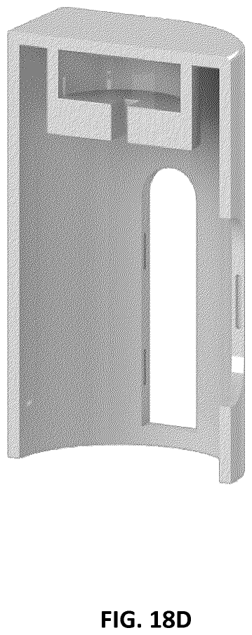

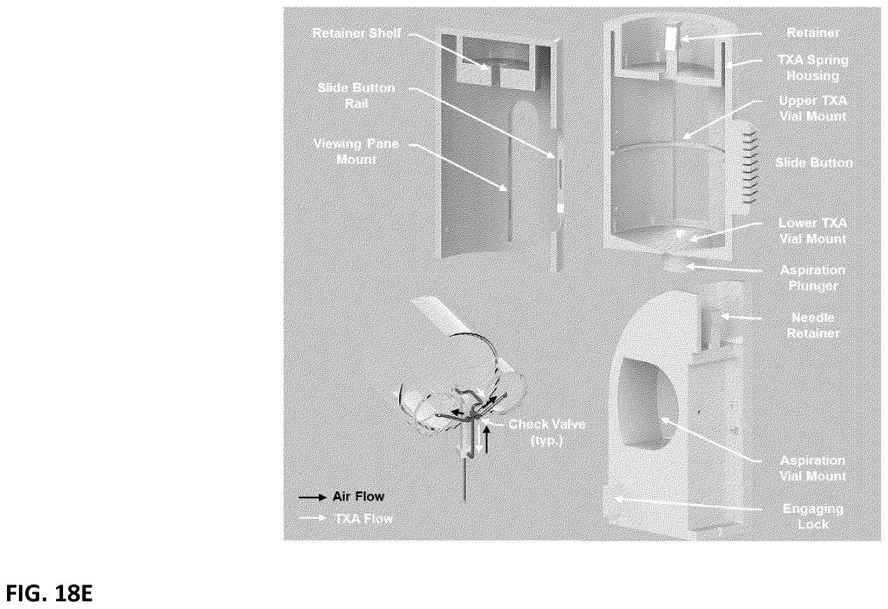

With reference now to FIG. 18, the various components of the delivery device 200 and IO access device 300 of FIG. 17A are generally shown. The delivery device 200 generally includes a catch mechanism 201, an outer cartridge 220, an inner cartridge 240 configured to be movably disposed within the outer cartridge 220, aspiration plunger 259, a first actuator 260, window 280a, an energy storage member 310, a plunger assembly 330, a releasable retainer member 340, a fluid container 350, an aspiration container 370. The IO access device generally includes a housing 310, an engaging lock 312, side cut-out sections 313, a second actuator 410 configured to be releasably coupled to the coupling end 450, a driver 430 and a tissue penetrator 470 configured to be releasably coupled to coupling end 450. The fluid delivery system 350 (not shown) may be positioned within the delivery device 200 or the IO access device 300. The delivery device 200 may be connected to the IO access device 300 as described above and the delivery device and IO access device may be operated in a substantially similar manner as described above.

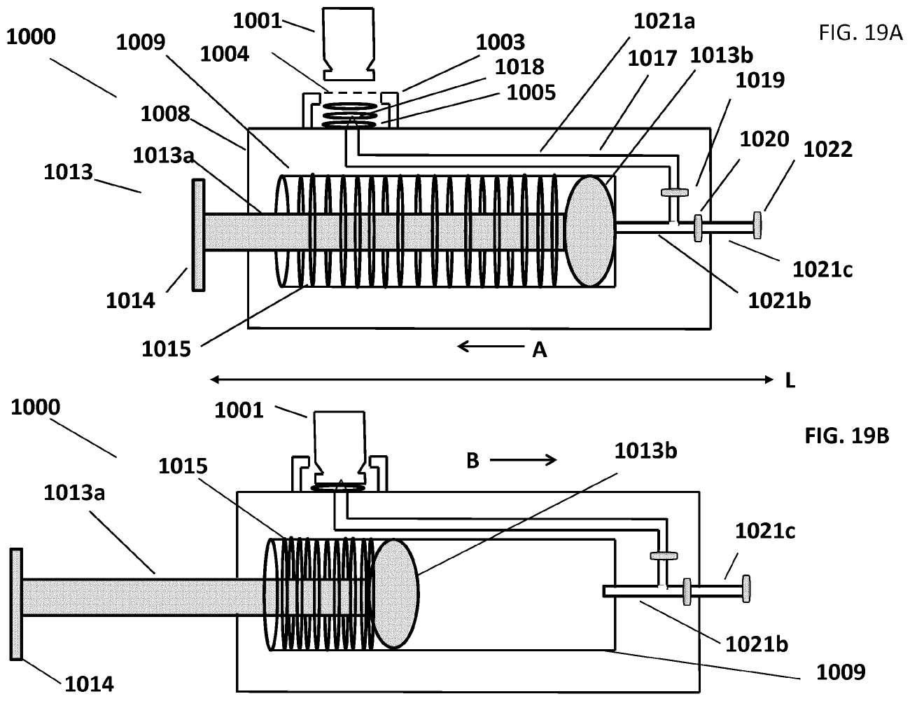

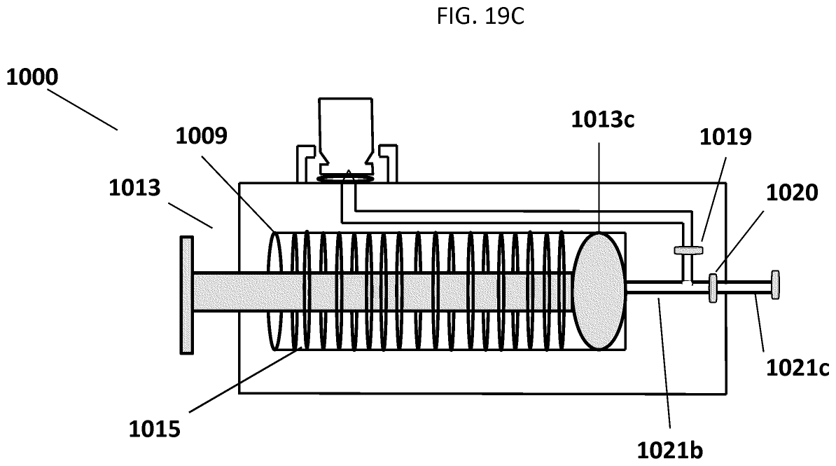

With reference now to FIGS. 19A, 19B and 19C, a cross-sectional view of a delivery device 1000 is shown according to another embodiment in an initial position, in a first configuration and in a second (advanced) configuration, respectively. In general terms, the delivery device 1000 includes an outer cartridge or casing 1008. The outer cartridge 1008 includes a proximal end having an opening configured to receive a plunger assembly 1013 and allow the plunger assembly to be movably disposed therein and a distal end comprising an opening or outlet. The outer cartridge 1008 includes an outer housing 1003 configured to hold a fluid container 1001 (filled or partially filled with a fluid, such as the medicaments described above, and which may be a standard vial known to those skilled in the art) on the outer surface of outer cartridge 1008. Outer housing 1003 may include a cap 1004 and a spring 1005 disposed within the outer housing 1003. Outer cartridge 1008 is further configured to hold an inner cartridge 1009.

The inner cartridge 1009 has a proximal end having an opening configured to receive the plunger assembly 1013 and a distal end comprising an orifice or outlet. The plunger assembly 1013 is configured to be movably disposed within the inner cartridge 1009.

The delivery device 1000 also includes an energy storage member 1015 operable to drive the plunger assembly 1013 in a distal direction, an actuator 14 operable to activate the delivery device 1000 and a fluid delivery system 1017.

The fluid delivery system 1017 includes an inlet configured to fluidly connect to the outlet of the fluid container 1001, a seal penetrator 1018 configured to rupture the seal on the outlet of the fluid container 1001 when the fluid container is placed within the outer housing 1003, a one-way valve 1019, a flow regulator or plate 1020 and an outlet 1022 configured to fluidly connect to an IO access device or IV line positioned within the IO space of the subject by a connector, such as a luer lock. The fluid delivery system 1017 further includes a first conduit 1021a fluidly coupled between the outlet of the fluid container 1001 and inlet of the one-way valve 1019, a second conduit 1021b fluidly coupled between the outlet of the one way valve 1019 and the outlet of the inner cartridge 1009 and a third fluid conduit 1021c fluidly coupled between the regulator valve or orifice plate 1020 and outlet 1022. First, second and third fluid conduits 1021a, 1021b, 1021c may be sterile flexible tubing.

The delivery device 1000 can generally be operated by removing cap 1004 (which may be removably attached) of the outer housing 1003, placing the fluid container 1001 into the outer housing 1003 and pressing down on the fluid container 1001 to break the seal at the outlet of the fluid container 1001 via the seal penetrator 1018. In another embodiment, the cap is not removably attached, but instead may be a donut-shaped cover that guards the seal penetrator when not in use. When delivery device 1000 is in its resting state, the donut-shaped cover will be positioned at the top of the outer housing 1003 due to a force produced by the spring. When the fluid container 1001 is then pressed down towards the seal penetrator 1018, the donut-shaped cover and spring will also be pressed down, exposing the seal penetrator 1018. Accordingly, when the fluid container 1001 is subsequently removed, the donut-shaped cover can be forced back up to the top of the outer housing 1003 by the force produced by the spring to guard the seal penetrator 1018.

The delivery device 1000 can then be activated by activating the actuator 1014 to cause a sequence of movements whereby the plunger assembly 1013 is displaced within the inner cartridge 1009 in a proximal direction from its initial position to a first position to draw the fluid contained within the fluid container 1001 through the first and second conduits 1021a, 1021b and into the inner cartridge 1009. The actuator 1014 can then be subsequently released and the plunger assembly 1013 can be displaced in a distal direction within the inner cartridge 1009 from its first position to a second (or advanced) position by the energy storage member 1015 to force the fluid confined within the inner cartridge 1009 through the second fluid conduit 1021b, flow regulator or plate 1020 and third fluid conduit 1021c at a relatively constant volumetric flow rate. As discussed above, the outlet of the third conduit 1021c may be fluidly connected to an IO access device implanted into the IO space of a subject (not shown) using any known type of connector (for e.g. luer lock).

The actuator 1014 may be any suitable device for activating the delivery device 1000, such as, for example, a handle. Upon activation, a plunger rod 1013a of plunger assembly 1013 is displaced within the inner cartridge 1009, as indicated by arrow A in FIG. 19A, along a longitudinal axis L between an initial position (FIG. 19A) and a first position (FIG. 19B). As illustrated, when the plunger rod 1013a is displaced in a proximal direction from its initial position to first position, a plug 1013b of plunger assembly 1013 is also moved in the proximal direction from its initial position to first position within the inner cartridge 1009 thereby drawing fluid contained within the fluid container 1001 into the inner cartridge 1009. One-way valve 1019 is configured to completely prevent the backflow of any fluid drawn into the inner cartridge 1009.

Once the inner cartridge 1009 is sufficiently filled with the fluid contained within the fluid container 1001, the energy storage member 1015 can be deployed by releasing the actuator 1014. As illustrated, the plug 1013b of plunger assembly 1013 is then moved in a distal direction, as indicated by arrow B in FIG. 19B, along a longitudinal axis L by the energy storage member 1015. Thus, when the plunger assembly 1013 is in its first (or retracted) position, the plug 1013b of plunger assembly 1013 is positioned at the proximal end of inner cartridge 1009. When the plunger assembly 1013 is moved to its second position (FIG. 19C), the plug 1013b is advanced from its first position at the proximal end of the inner cartridge 1009 towards its second position at the distal end of the inner cartridge 1009. In this manner, as the plug 1013b is advanced, fluid is conveyed from within the inner cartridge 1009 into the IO space of a subject via the second conduit 1021b and third conduit 1021c at a relatively constant volumetric flow rate using the force produced by the energy storage member 1015.

The energy storage member 1015, shown as a spring, also defines longitudinal axis L. The energy storage member 1015 can be moved within the inner cartridge 1009 along the longitudinal direction L between a first configuration (FIG. 19B) and a second configuration (FIG. 19C). When the energy storage member 1015 is in its first configuration, the energy storage member 1015 has a first potential energy. When the energy storage member 1015 is in its second configuration, the energy storage member 1015 has a second potential energy that is less than the first potential energy. The energy storage member 1015 is operably connected to the plunger assembly 1013 such that when the energy storage member 1015 moves from its first configuration to its second configuration, it converts at least a portion of its first potential energy into kinetic energy to move the plunger rod 1013a and plug 1013b of plunger assembly 1013 between their first position and second position. Said another way, the movement of the energy storage member 1015 from its first configuration to its second configuration results in the production of a force that acts upon the plug 1013b of the plunger assembly 1013 to move the plug 1013b between its first position and second position and thereby dispense fluid contained within the inner cartridge 1009 through the second conduit 1021b and third conduit 1021c at a relatively constant volumetric flow rate. A desired flow of fluid (i.e. relatively constant volumetric flow rate) that is being dispensed into the third conduit 1021c can be achieved using any type of known flow regulator, such as a variable adjustable valve, or plate (i.e. orifice plate) 1020 which can be positioned between the second fluid conduit 1021b and third fluid conduit 1021c. One-way valve 1019 again prevents the backflow of any fluid being dispensed through the second conduit 1021b.

The present disclosure also provides a wide variety of kits, devices and associated components which may be used to obtain vascular access to the subject. In some embodiments, such kits may include the delivery device according to the present disclosure, an IO access device, one or more connectors to communicate fluids with the subject's IO space, a fluid container containing a fluid and any combinations thereof. Such kits may also include apparatus which allows monitoring a subject.

Kits incorporating teachings of the present disclosure may be rigid, semi-rigid or soft-sided. Such kits may provide a convenient way to carry various components and devices operable to achieve vascular access in an organized and systematic fashion. Such kits may present EMS first responders and other medical personnel with a well-organized collection of components and devices to achieve vascular access by placement of IV catheters and/or IO catheters. For some embodiments, a kit incorporating teachings of the present disclosure may be combination an IV kit, an IO kit and/or a unit dose kit in one convenient bag.

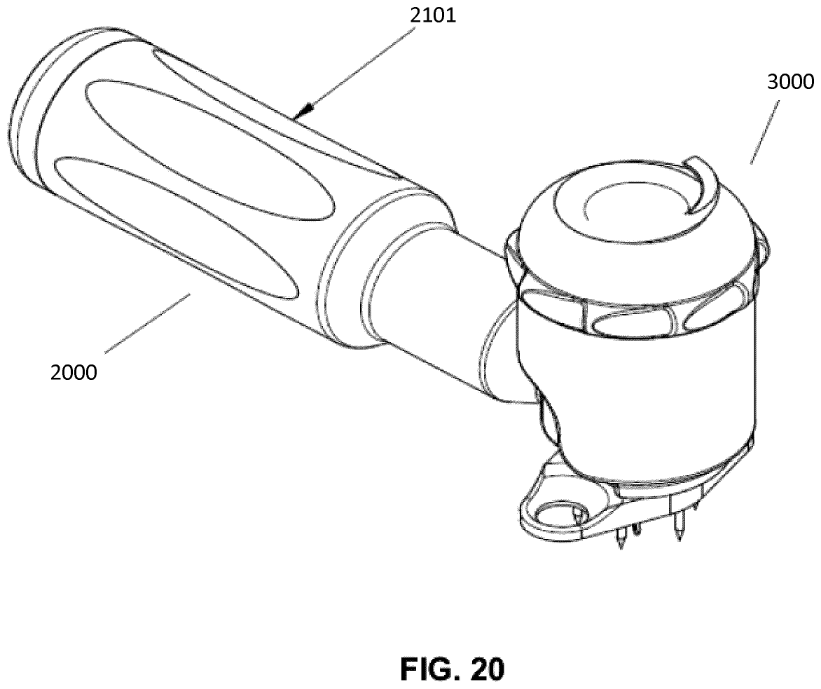

With reference now to FIG. 20 a particular delivery device 2000 and IO access device 3000 according to embodiments described above is shown, the delivery device and the IO access device disposed in an outer cartridge 2101. The delivery device and IO access device may contain one or more features described in the embodiments above.

In some embodiments, delivery device 2000 and IO access device 3000 may be operable when aligned with either the left or right collarbone and thus are operable in two different orientations.

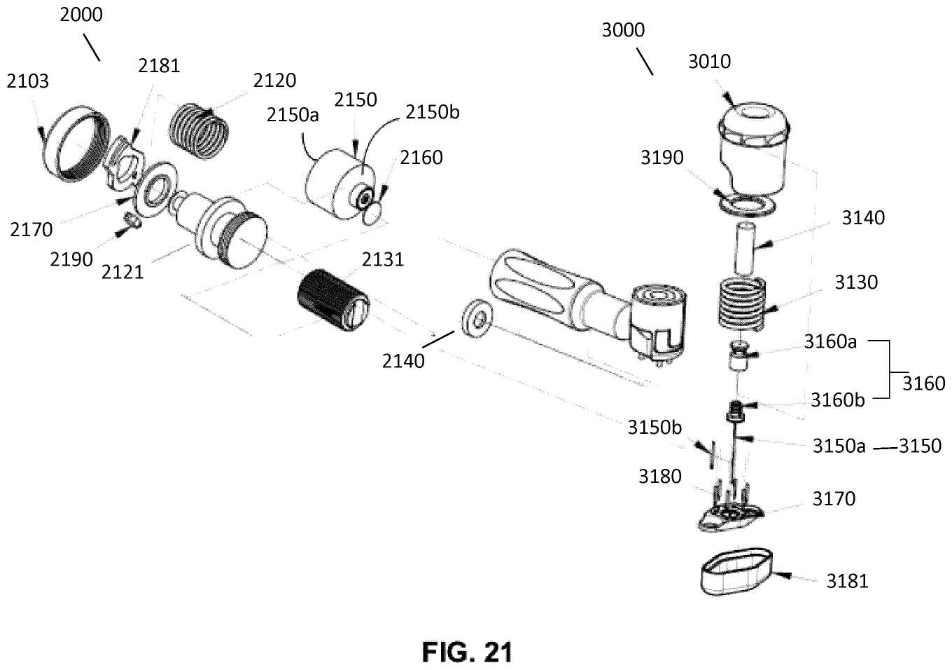

With reference to FIGS. 21, 24B and 27, the delivery device 2000 generally includes an energy storage member 2120 that is configured to, once activated, move from a first configuration to a second configuration and provide a force in the distal direction, a plunger assembly 2121 configured to move from a first position to a second position when the energy storage member is activated and moves from its first configuration to second configuration, flow regulator 2131, washer 2140, fluid container 2150 sized and configured to be placed between the plunger assembly and flow regulator and having an open proximal end 2150a and a closed distal end 2150b with an orifice or outlet, and diaphragm 2160 sized and configured to be initially be in a closed configuration to hold fluid inside of fluid container 2150 and an open configuration once pressure is applied to the fluid within the fluid container by the plunger assembly 2121 as it moves from its first position which may be at or near the proximal end 2150a of fluid container 2150 to its second position which may be at or near the distal end 2150b of fluid container 2150. In some embodiments, diaphragm 2160 is located at the distal end 2150b of fluid container 2150, and in others it is mounted concentrically within the interior space of outer cartridge 2101. In some embodiments, energy storage member 2120 may be constantly acting on and applying a force to plunger assembly 2121 and translation of plunger assembly 2121 from the first position to the second position is prevented by back pressure created by a temporary fluid seal created at an interface between the distal end of the delivery device and the bottom of fluid channel assembly 3160. In some embodiments, the delivery device 2000 further includes an actuator 2181 which prevents the energy storage member 2120 from being activated and providing the force causing plunger assembly 2121 to move from the first position to the second position until the actuator 2180 has been activated. Actuator 2180 can be comprised of any suitable mechanism including a button, a pin, or similar mechanism described above. In other embodiments, actuator 2180 contains retaining ring 2170 and button 2190 which prevents the actuator 2180 from being activated unintentionally.

Continuing to refer to FIG. 21, IO access device 3000 is in fluid communication with the delivery device 2000 and generally contains a second actuator 3010 which, when activated, is configured to translate or slide downwards and is also configured to be removed after the IO space has been accessed, an energy storage member 3130 operable and configured to provide an upward force to remove the actuator 3010 once a torque is applied to the actuator 3010, a tissue penetrator assembly 3150 including a trocar 3150a and a cannula 3150b, a driver 3140 coupled to the tissue penetrator assembly 3150, such as to the trocar 3150a, and configured to be movably disposed from a first position to a second position within outer cartridge 2101 when the second actuator 3010 is activated, fluid channel assembly 3160 which is coupled to the tissue penetrator assembly, such as the cannula 3150b, and configured to move in a downwards direction (negative z-direction) when the driver 3140 moves from its first position to second position, base 3170, and sounding needle array 3180. The trocar 3150a is configured to provide structural support to the cannula 3150b during insertion and can be made from any suitable biocompatible plastic or metal and which can be later removed to allow for fluid flow. Cannula 3150b can be any suitable open cylindrical channel and may be flexible or rigid and is made from a biocompatible material. Fluid channel assembly 3160 includes an upper channel assembly 3160a and lower channel assembly 3160b. In some embodiments, upper channel assembly 3160 is fitted with a luer-lock, or other suitable connection apparatus at its upper surface through which secondary fluids can be administered. In some embodiments, IO access device 3000 further contains a safety mechanism 3190 to prevent the second actuator 3010 from being activated and translating downwards until in use. This can be any suitable safety mechanism known to those skilled in the art. Finally, in some embodiments, IO access device 3000 further contains a needle sheath 3181 to prevent needlestick injuries by providing a protective barrier around sounding needle array 3180. The needle sheath can be manually removed or may automatically remove itself during insertion of tissue penetrator assembly 3150 into the skin of the subject, and re-cover sounding needle array 3180 after use.

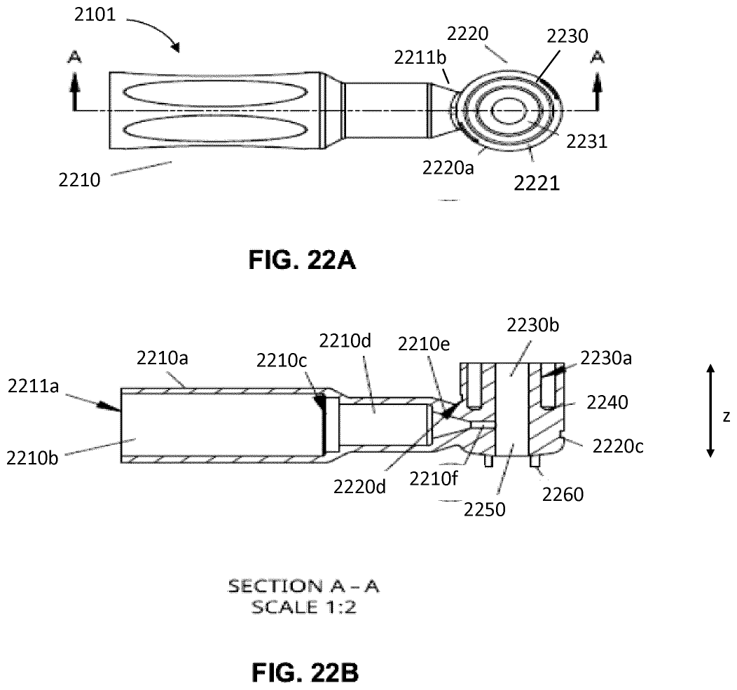

With reference now to FIGS. 22A and 22B, outer cartridge 2101 is generally shaped and dimensioned to fit within an operator's hand and includes outer wall 2210 with an exterior surface 2210a and an interior surface defining multiple interior spaces, 2210b through 2210f. While FIGS. 22A and 22B depicts that outer cartridge 2101 exists as a single, one-piece structure, it will be understood that according to other embodiments outer cartridge 2101 may be separated into multiple components and such components may be joined via a rigid or flexible connection. In some embodiments, wall 2210 will have first and second elongated windows configured to allow an operator to view the contents within delivery device 2000 such as described above. These windows may be covered with clear material, such as a translucent or transparent material, to maintain sterility of delivery device 2000 while allowing the operator to view the contents of the system. Outer cartridge 2101 is configured to house delivery device 2000 and at least a portion or substantially all of IO access device 3000. While a unitary (i.e. one-piece) wall 2210 has been illustrated in FIGS. 22A and 22B that defines both exterior surface 2210a and interior surface defining interior spaces 2210b, 2210c, 2210d, 2210e, and 2210f, it will be understood that according to other embodiments wall 2210 may include a plurality of layers with different layers defining the exterior and interior surface. Interior space 2210b is sized and configured to hold fluid container 2150. Interior space 2210c is sized and configured to house washer 2240a. Interior space 2210d is sized and configured to house flow regulator 2131. Interior spaces (or fluid channels) 2210e and 2210f are sized and configured to enable fluid from the fluid container to flow into IO access device 3000. At the proximal end of outer cartridge 2101, an opening is shown which in some embodiments couples with an end cap 2103. In other embodiments, end cap 2103 is part of outer cartridge 2101. In some embodiments, energy storage member 2120 is directly adhered to end cap 2103. In other embodiments, where end cap 2103 is part of outer cartridge 2101, energy storage member 2120 is directly adhered to outer cartridge 2101. Still in other embodiments, end cap 2103 or outer cartridge 2101 features specific mounting points at which energy storage member 2120 may be attached to.

Energy storage member 2120 may comprise any suitable means of power generation to provide a force, such as, one or more of the following: a battery, a spring, compressed gas, and any other mechanical or electrical source of rotation or reciprocation. In addition to batteries, electric power may come from any other suitable source including conventional wall outlets. The power source may be operably coupled with a motor. Motors may be selected from the group consisting of DC motors, AC motors, compressed gas motors, wound spring motors, and reciprocating motors. Motors may also be coupled to one or more gears, which may optionally be positioned in one or more gear boxes.

In some embodiments, no air will exist in interior spaces 2210d, 2210e, and 2210f, and these spaces will be pre-charged with fluid. This fluid may be a medicament or normal saline. In other embodiments, the interior spaces 2210d, 2210e, and 2210f will be filled with air.

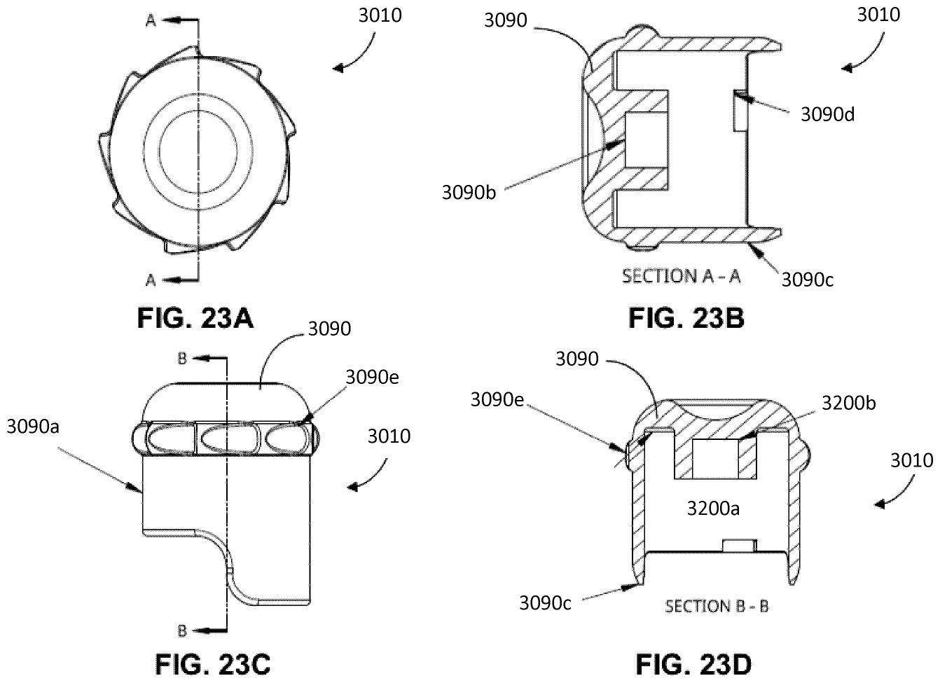

Moving distally, outer cartridge 2101 is configured to house at least a portion of IO access device 3000 (for e.g. substantially all of IO access device except actuator 3010 which is configured to translate downwards or slidably disposed over the outer cartridge when activated) within walls 2220 and 2230. While a unitary (i.e. one-piece) wall 2220 has been illustrated in FIGS. 22A and 22B that defines both exterior surface 2220a and interior surface 2220b it will be understood that according to other embodiments wall 2220 may include a plurality of layers with different layers defining the exterior and interior surfaces. Wall 2220 further includes rails 2220c and 2220d, through which catches 3090c, 3090d (see FIG. 23B) translate, and flat feature 2221. Again, moving distally, outer cartridge 2101 further includes inner wall 2230. Inner wall 2230 includes exterior surface 2230a and interior surface 2230b. Driver 3140 is sized and configured to fit within the diameter of interior surface 2230b and slide in the z-axis direction through it. Stop 2231 prevents second actuator 3010 from being pushed or translating too far downwards and tissue penetrator assembly 3150 from over-extending. Outer cartridge 2101 further includes stop 2240 which acts as one anchor point for energy storage member 3130. Bottom end 2250 and base mount 2260 are configured to connect directly to base 3170 to form a temporarily rigid connection which can be disconnected, such as after fluid delivery to the subject.

According to certain embodiments of the present disclosure, the walls 2110, 2220, and 2230 may be rigid. According to other embodiments, the walls 2110, 2220, and 2230 may be flexible, whether according to the nature of the material that defines the walls or according to the nature of the structure of the walls. The walls 2110, 2220, and 2230 may be made of glass, metal, or polymer, for example. In particular, polymer versions may be made of polycarbonate, polypropylene, polyethylene (such as high density polyethylene), polytetrafluoroethylene, cyclic olefin polymer, cyclic olefin copolymer, crystal zenith olefinic polymer, nylon, or engineering resins. As to flexible versions of the walls 2110, 2220, and 2230, butyl rubber, silicon-based rubber, latex-based rubber, coated rubber, as well as multi-layer polymer films, such as polyethylene (such as low density polyethylene) and polypropylene, may be used.

The wall 2110 may have a generally stepped cylindrical shape (although other shapes are contemplated, such as rectangular). According to some embodiments, the wall 2110 may have a length of between about 70 mm to about 160 mm and defines two opposed, open ends, proximal end 2111a and distal end 2111b. Interior space 2210b may have a length between 60 and 100 mm. Interior space 2210c may have a length between 5 and 20 mm. Interior space 2210d may have a length between 20 and 60 mm. Interior space 2210e may have a length between 10 and 30 mm. Finally, interior space 2210f may have a length between 5 and 20 mm.

The interior spaces of wall 2110 of outer cartridge 2101 are configured to hold several parts in place. Interior space 2210b is configured to hold fluid container 2150. Interior space 2210b may be further adapted and configured to have specific mounting points for fluid container 2150 or fluid container 2150 may be adhered to the surface of space 2210b in a suitable manner. In some embodiments, fluid container 2150 does not translate relative to outer cartridge 2101 in any axis-direction. Interior space 2210c is sized and configured to hold washer 2140. Interior space 2210d is sized and configured to hold flow regulation apparatus 2131. In some embodiments, interior space 2210f is sized and configured to hold diaphragm 2160, while in others diaphragm 2160 will be located at the distal end of fluid container 2150.

As described above and referring to FIG. 21, the delivery device is configured to include and hold a fluid container 2150 within the outer cartridge 2101 in interior space 2210b. The fluid container includes a proximal end 2150a and a distal end 2150b that is fluidly coupled to washer 2140 and flow regulator 2131. In some embodiments, the fluid in fluid container 2150 comprises a medicament, such as, for example, tranexamic acid or any of the other medicaments described above. The fluid container 2150 may contain a volume of about 1 mL to about 20 mL and have a length between 10 mm and 40 mm. Both the proximal and distal ends may have a diameter of about 25 mm to about 40 mm. The orifice of the distal end may have a diameter of between about 0.05 mm to about 1.6 mm.

Also, as described above, delivery device 2000 is configured to include flow regulator 2131. Flow regulator 2131 includes a proximal end, a distal end and a helical channel. Flow regulator 2131 is designed to form a fluid-tight seal with interior space 2210d about its diameter. Thus, fluid will only flow through helical channel and not between coils of the helix. The configuration of the helical channel can be altered to produce different head losses through flow regulator, lowering or raising the volumetric fluid flow rate of delivery device 2000. This variation in volumetric flow rate can be achieved by changing the length of the channel, or by changing the cross-sectional area of the channel. In some embodiments, the channel features a semi-elliptical cross-section, whereas in others it may feature a rectangular cross-section, a semi-circular cross-section, a triangular cross-section, or any other suitable cross-section. In one embodiment, the helical channel provides sufficient head loss and is matched to the force produced by the energy storage member 2120 such that fluid flow is maintained at a relatively low and pseudo-constant volumetric flow rate over a long duration infusion of fluid or medicament to the subject. The flow regulator 2131 may also include tubing wrapped around the helical channel (not shown).

Referring to FIGS. 22A and 22B, the wall 2220 may have a generally cylindrical shape (although other shapes are contemplated, such as rectangular). According to some embodiments, the wall 2220 may have a length between 20 and 70 mm. The diameter of interior surface 2220b is designed to be slightly less than the diameter of exterior surface 2230b. The diameter of exterior surface 2220a is between 20 and 60 mm.

Base 3170 is configured to be mounted directly to outer cartridge 2101 using base mount 2260. In some embodiments, this connection is formed via a dowel system as shown in FIG. 22B. In other embodiments, this connection is formed via a press-fit, a mild adhesive, a screw fit, or any other suitable connection method. However, in all embodiments, this connection is a temporary, removable connection that is broken after the fluid or medicament has been fully delivered to the IO space. This will be expanded upon further in subsequent step-by-step use instructions described below.

Base 3170 is further fitted with sounding needle array 3180. Sounding needle array 3180 is configured to penetrate the skin above the manubrium first and is the first part of IO access device 3000 which contacts the manubrium during use.

Base 3170 also features a landmarking system which enables the operator to find the correct site of insertion in a repeatable fashion. The site of insertion is the manubrium. This landmarking system may feature cylindrical cut-outs that are used in tandem with the operators thumb or fingers. This landmarking system will be expanded on in step-by-step use instructions below. It may feature any other sufficient landmarking system that ensures repeatability.

Referring to FIGS. 22A and 22B, the inner wall 2230 may have a generally cylindrical shape (although other shapes are contemplated, such as rectangular). According to some embodiments, the wall 2230 may have a length equal to that of the length of wall 2220. Interior surface 2230b and exterior surface 2230a are sized and configured in such a way that energy storage member 3130 can be housed between them. Interior surface 2230b is sized and configured to allow driver 3140 to translate in the z-axis (or perpendicular to the movement of the plunger assembly 2121). The diameter of interior surface 2230b is between 5 mm and 20 mm.

With reference now to FIGS. 23A-D, second actuator 3010 is generally shaped and dimensioned to fit within an operator's hand, while the operator is applying a downward force in the z-axis with the palm and also when the same operator is grabbing the top of second actuator 3010 and twisting for removal of second actuator 3010. Second actuator 3010 includes a wall 3090 with exterior surface 3090a and interior surface 3090b. While a unitary (i.e., one-piece) wall 3090 has been illustrated in FIGS. 23A-D that defines both the exterior and interior surfaces 3090a, 3090b, it will be understood that according to other embodiments the wall 3090 may include a plurality of layers with different layers defining the exterior and interior surfaces 3090a, 3090b. Wall 3090 may further include catches 3090c and 3090d, which translate through aforementioned rails 2220c and 2220d. On exterior surface 3090a, wall 3090 has groove 3090e. Groove 3090e is used for operator grip during removal of second actuator 3010 and may instead be substituted for any appropriate structure that ensures grip while applying torque to second actuator 3010. In some embodiments, wall 3090 features a contour on its top surface to further assist with operator grip.

According to certain embodiments of the present disclosure, the wall 3090 may be rigid. According to other embodiments, the wall 3090 may be flexible, whether according to the nature of the material that defines the walls or according to the nature of the structure of the walls. The wall 3090 may be made of glass, metal, or polymer, for example. In particular, polymer versions may be made of polycarbonate, polypropylene, polyethylene (such as high density polyethylene), polytetrafluoroethylene, cyclic olefin polymer, cyclic olefin copolymer, crystal zenith olefinic polymer, nylon, or engineering resins. As to flexible versions of the wall 3090, butyl rubber, silicon-based rubber, latex-based rubber, coated rubber, as well as multi-layer polymer films, such as polyethylene (such as low density polyethylene) and polypropylene, may be used.

The wall 3090 may have a generally cylindrical shape (although other shapes are contemplated, such as rectangular). According to some embodiments, the wall 3090 may have a length between 40 mm and 70 mm on its long side, and 20 mm to 50 mm on its short side. The diameter of interior surface 3090b is designed to be slightly greater than the diameter of exterior surface 2120a.

Defined by interior surface 3090b is an inner cylindrical section 3200, having an exterior space 3200a and interior space 3200b. Exterior space 3200a is designed to match the diameter of exterior surface 2130a, allowing the inner diameter of energy storage member 3130 to fit concentrically around it. Interior space 3200b is designed to hold driver 3140 in place and translate with it as it moves from the first position to the second position.

In some embodiments, interior surface 3090b is directly adhered to energy storage member 3130. In other embodiments, energy storage member 3130 is mounted to second actuator 3010 in another suitable manner. This allows for energy storage member 3130 to exert a force on second actuator 3010 until the actuator is removed.

Second actuator 3010, energy storage member 3130 and driver 3140 allow for transmission of operator force from the hand to the tissue penetrator assembly and insertion of the tissue penetrator assembly into the IO space. During operation, energy storage member 3130 acts to counteract the applied operator force to ensure that deployment of tissue penetrator assembly 3150 does not occur before IO access device 3000 contacts the manubrial surface via sounding needle array 3180. This will be expanded on further in subsequent step-by-step discussions of the operation of delivery device 2000. Energy storage member 3130 may comprise any suitable means of power generation, such as, one or more of the following: a battery, a spring, compressed gas, and any other mechanical or electrical source of rotation or reciprocation. In addition to batteries, electric power may come from any other suitable source including conventional wall outlets. The power source may be operably coupled with a motor. Motors may be selected from the group consisting of DC motors, AC motors, compressed gas motors, wound spring motors, and reciprocating motors. Motors may also be coupled to one or more gears, which may optionally be positioned in one or more gear boxes.

Second actuator 3010 is a removable feature with multiple opposing forces holding it in place until removal is desired. The first force on second actuator 3010 stems from energy storage member 3130 which exerts an upward force in the positive z-direction. Two reactionary forces from the interaction of catches 3090c and 3090d with rails 2120c and 2120d provide the opposing force in the negative z-direction. After accessing the IO space, the operator applies a torque to second actuator 3010. This allows both catches to translate along their rails until they are released. The release of catches 3090c and 3090d from rails 2120c and 2120d removes the force opposing energy storage member 3130 allowing the energy storage member to exert an upward force on the second actuator 3010 to remove the second actuator 3010 from the IO access device 3000.

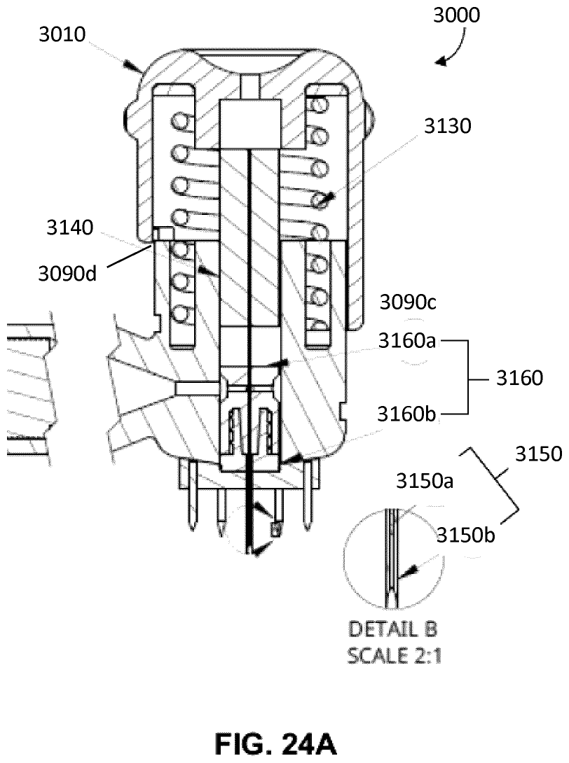

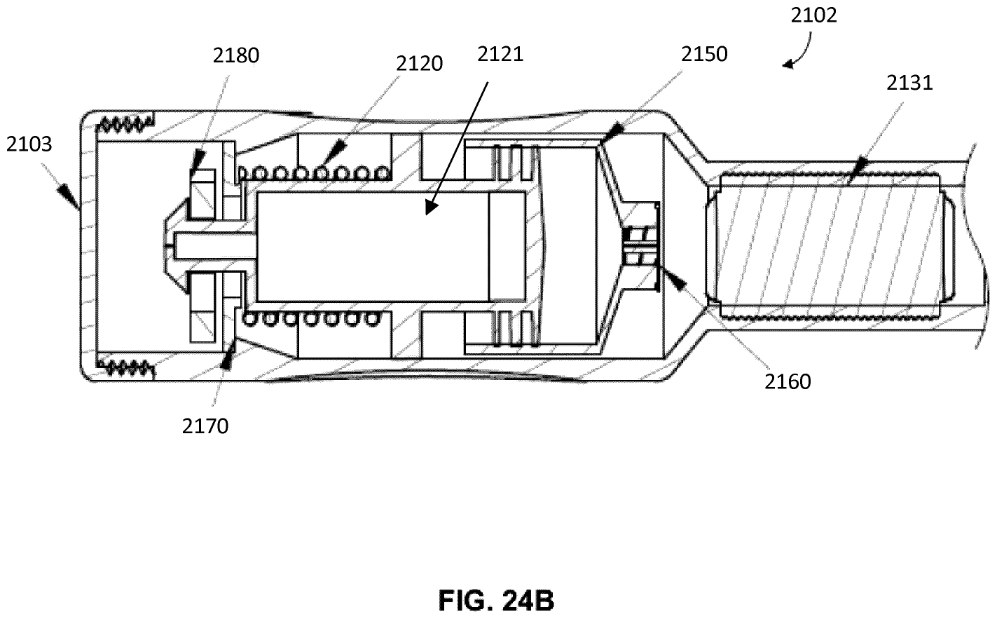

To illustrate operation of the delivery device 2000, a sequence of events illustrating accessing the IO space by the IO access device 3000 and then delivery of fluid by delivery device 2000, as well as the position of various components, is discussed and depicted in FIGS. 24A-28B.

FIGS. 24A and 24B illustrate delivery device 2000 and IO access device 3000 in their initial positions. In some embodiments, there exists a 2-step activation mechanism where driver 3140 is held in place by safety mechanism (not shown). For this discussion of the sequence of events, this mechanism will be omitted. In all embodiments, driver 3140 is held in place by opposing forces from energy storage member 3130 and the interactions between catches 3090c and 3090d, and rails 2220c and 2220d. Second actuator 3010, energy storage member 3130, driver 3140, and trocar 3150a of the tissue penetrator assembly 3150 are all operatively coupled to one another and will move as one once the operator applies force to the second actuator 3010 in the negative z-direction. Fluid channel assembly 3160 is operatively coupled to cannula 3150b of tissue penetrator assembly 3150 and is at its highest point at rest, where the top surface of upper fluid channel assembly 3160a is in contact with the bottom surface of driver 3140. Fluid channel assembly 3160 is blocking fluid flow through interaction with the distal end of the delivery device. Tissue penetrator assembly 3150 is housed entirely within IO access device 3000. Base 3170 is temporarily connected to outer cartridge 2101 via base mount 2260.

Continuing on, plunger assembly 2121 is in a first position, where the plunger tip is mounted concentrically within fluid container 2150. In some embodiments, the fluid pathway from fluid container 2150 to fluid channel assembly 3160 is pre-charged with fluid, whereas in others it will be pre-charged with air. For this discussion, it be assumed to be pre-charged with fluid, such as a medicament. Diaphragm 2160 is in its closed configuration and awaiting to be opened with a sufficient pressure differential created by movement of plunger assembly 2121. Energy storage member 2120 is at its most compressed state and providing a constant force on plunger assembly 2121. Movement of plunger assembly 2121 is restricted due to back pressure created by the interaction of fluid channel assembly 3160 and distal end of the delivery device. Once this interaction is broken after driver 3140 pushes fluid channel assembly 3160 in the negative z-direction, plunger assembly 2121 will be allowed to translate towards its second position and fluid or medicament will be able to flow from the fluid container 2150 to the fluid channel assembly 3160.

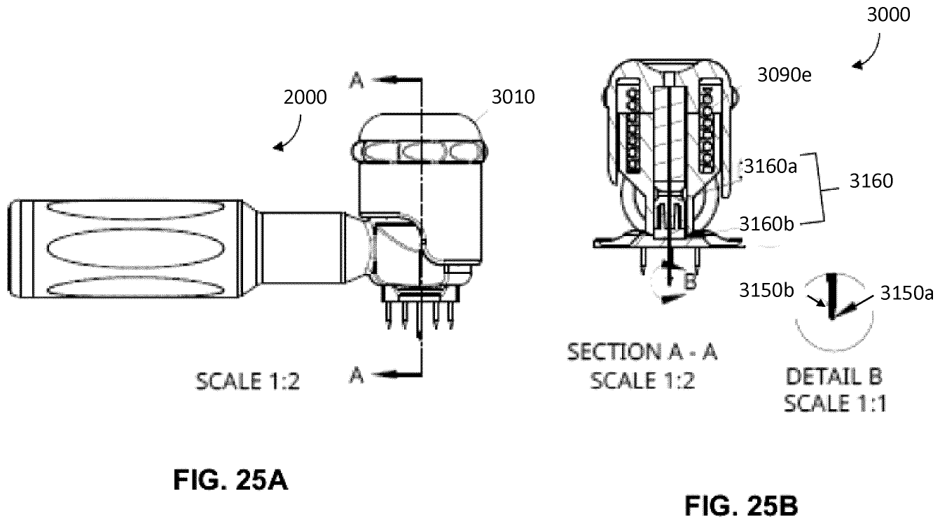

With reference to FIGS. 25A and 25B, to begin use of delivery device 2000 and IO access device 3000, the operator must first locate the correct location of insertion at the manubrium. To do so, the operator uses the landmarking system on base 3170. The operator aligns the top-most cut-out with the sternal notch above the manubrium, sticking their thumb or finger through the cut-out. The operator then aligns the other cut-out with the midsagittal plane. Once the correct point of insertion is found, the operator can activate IO access device 3000 by pushing down on second actuator 3010. The first part to penetrate the skin above the manubrium is sounding needle array 3180. It is important to note that second actuator 3010, driver 3140, trocar 3150a, cannula 3150b, and fluid channel assembly 3160 translate a minimal amount relative to outer cartridge 2101 until sounding needle array 3180 has contacted the manubrium. This ensures that premature activation of delivery device 2000 does not occur. Once contact occurs, second actuator 3010, driver 3140, trocar 3150a, cannula 3150b, and fluid channel assembly 3160 translate in the negative z-direction towards the subject. At the bottom of this slide, when fluid channel assembly 3160 is in contact with base 3170, the IO space at the manubrium is fully accessed by the tissue penetrator assembly.

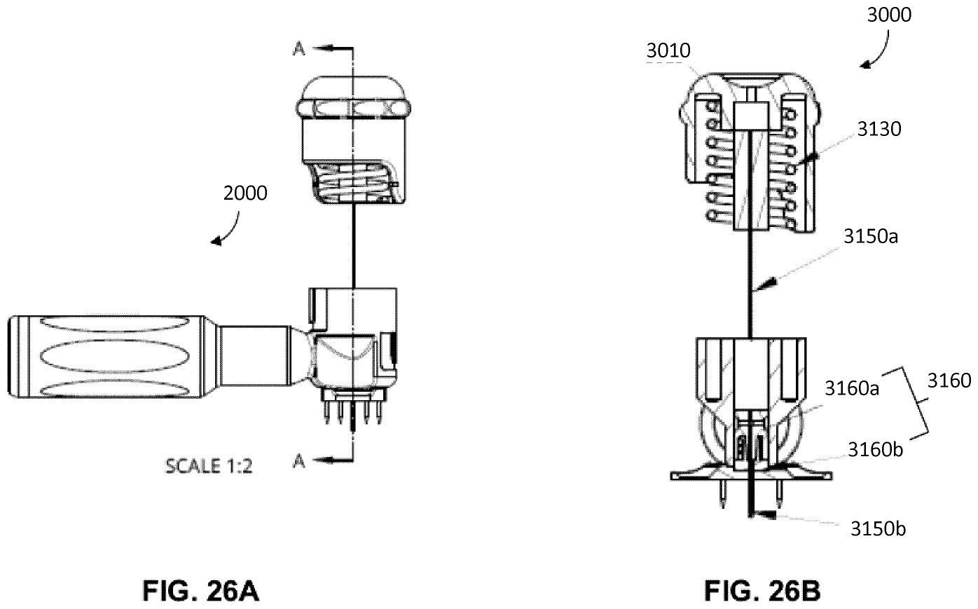

With reference to FIGS. 26A and 26B, delivery device 2000 is activated once fluid channel assembly 3160 descends towards the subject and contacts base 3170. This releases the blockage created at the interface by the interaction between distal end of delivery device and fluid channel assembly 3160. With the blockage removed, fluid can now flow into fluid channel assembly 3160 due to pressure/force created by energy storage member 2120 and acting on plunger assembly 2121. Diaphragm 2160 is also in an open configuration due to this change in pressure differential. However, fluid cannot yet flow to the IO space due to the presence of trocar 3150a inside of cannula 3150b. Second actuator 3010, driver 3140, and trocar 3150a must be removed to open cannula 3150b. To remove these components, the operator applies a torque to second actuator 3010, using groove 3090e for grip. This creates rotation of second actuator 3010, which removes catches 3090c and 3090d from the horizontal portion of rails 2220c and 2220d. Once removed, energy storage member 3130 can now push second actuator 3010, and corresponding connected components driver 3140 and trocar 3150a, up and away from outer cartridge 2101. The operator can then fully remove these components, opening the fluid pathway. It should be noted here that a luer lock, check valve, or other method of sealing the fluid pathway after trocar 3150a removal is located at the top of fluid channel assembly 3160 to ensure fluid does not escape after trocar 3150a removal. As noted above, in some embodiments the delivery device 2000 may include an actuator which prevents the unintentional activation of the delivery device.

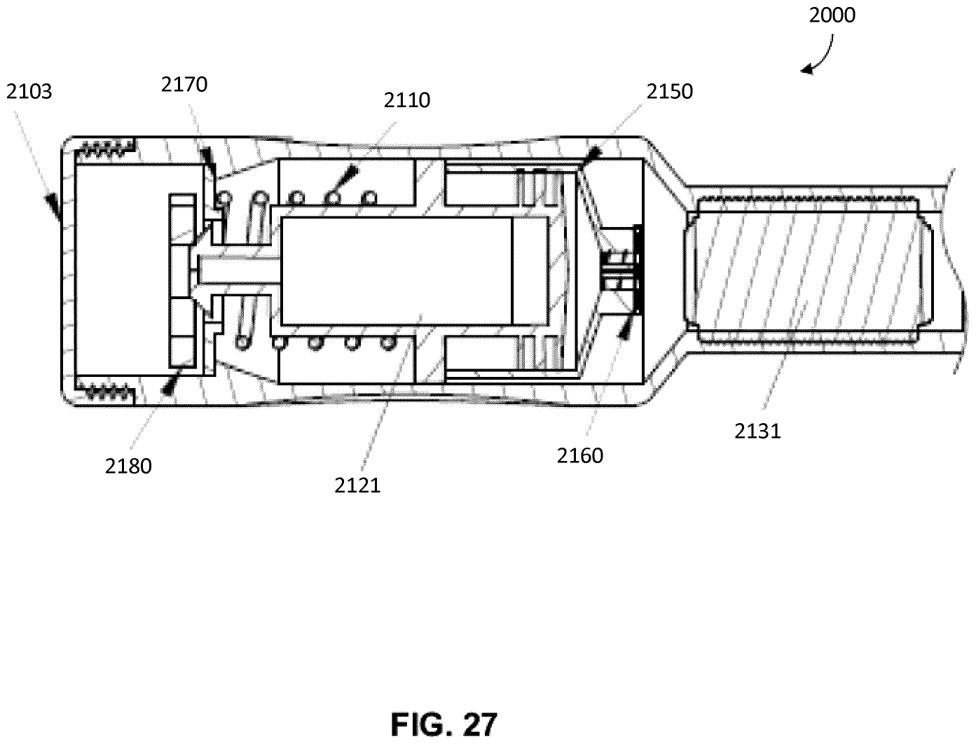

With reference to FIG. 27, now that trocar 3150a has been removed, fluid is allowed to flow from fluid container 2150 to the IO space. Plunger assembly 2121 and energy storage member 2120 work in tandem to produce a pressure differential which facilitates this flow. Flow regulator 2131 ensures that fluid flow is maintained at a relatively constant volumetric fluid flow rate as the plunger moves from its first position to its second position.

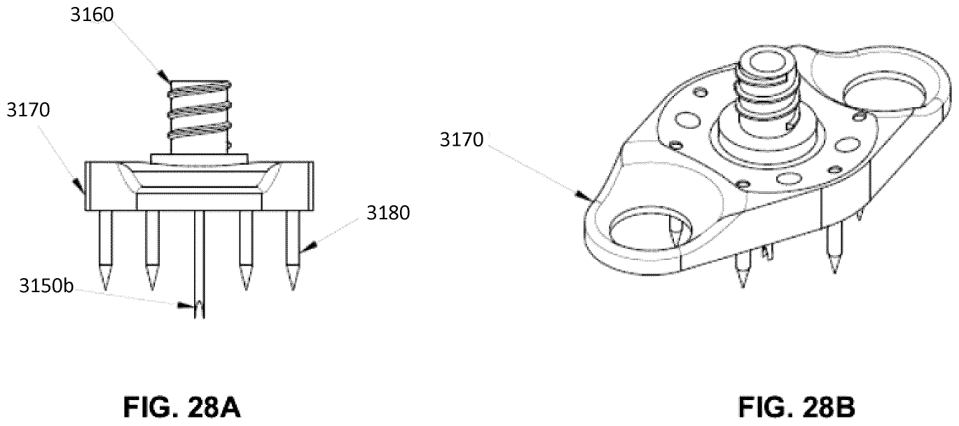

With reference to FIGS. 28A and 28B, fluid (i.e. medicament) has been fully provided to the subject and the plunger is at rest at its second position. If the operator desires to administer secondary fluids or medicaments, the operator can remove outer cartridge 2101 and it's contents, leaving behind fluid channel assembly 3160, cannula 3150b, base 3170, and sounding needle array 3180 by pressing down on base 3170 and pulling up on the outer cartridge at wall 2110. Secondary medicaments can then be administered through fluid channel assembly 3160, through a luer-lock or other suitable mechanism, as discussed above. Once secondary medications have been administered and treatment is complete, the operator removes all remaining components, re-covers sounding needle array 3180, and disposes of these materials as biohazardous.

What is claimed is:

1. A delivery device adapted to deliver a fluid into an IO space of a subject at a relatively constant volumetric flow rate over an extended period of time comprising:
(i) an outer cartridge;
(ii) an inner cartridge configured to be movably disposed in a proximal direction within the outer cartridge and configured to hold a fluid container containing the fluid;
(iii) an actuator operable to activate the delivery device;
(iv) a plunger assembly sized and configured to be movably disposed within the fluid container from a first position to a second position;
(v) an energy storage member operable to produce a force to drive the plunger assembly from in a distal direction from the first position to the second position after activation of the delivery device;
(vi) a releasable retainer member configured to secure the plunger assembly in the first position and to release the plunger assembly after activation of the delivery device; and
(vii) a fluid delivery system configured to fluidly couple to an outlet of the fluid container and to deliver the fluid contained in the fluid container to an IO access device or IV line positioned in the IO space of the subject
wherein in operation the delivery device is activated by activating the actuator to cause a sequence of movements whereby the inner cartridge is displaced within the outer cartridge in the proximal direction and the plunger assembly is subsequently released from the retainer member and displaced from the first position to the second position by the force produced by energy storage member to force the fluid confined in the fluid container to dispense through the fluid delivery system at the relatively constant volumetric flow rate to the IO access device or IV line and to the IO space of the subject.

2. The delivery device of claim 1, wherein the outer cartridge is configured to hold an aspiration container and the inner cartridge further comprises an aspiration plunger movably coupled to its distal end sized and configured to be movably disposed within the aspiration container in the proximal direction.

3. The delivery device of claim 2, wherein the fluid delivery system comprises a first conduit configured to fluidly couple to an outlet of the aspiration container and comprising a first one-way valve having a first outlet and a second conduit configured to fluidly couple to the outlet of the fluid container and comprising a second one-way valve having a second outlet and wherein the first outlet and the second outlet are fluidly coupled to form a third conduit configured to aspirate the aspiration container and to deliver the fluid contained in the fluid container.

4. The delivery device of claim 3, wherein the first one-way valve is configured to completely prevent backflow of air from the first conduit to the third conduit and wherein in operation when the inner cartridge is displaced in the proximal direction the aspiration plunger is movably disposed within the aspiration container in the proximal direction to aspirate the third conduit.

5. The delivery device of claim 1, wherein the relatively constant volumetric flow rate is achieved either by matching the force produced by the energy storage member with a diameter of the outlet of the fluid container or by a flow regulator or by an orifice plate.

6. The delivery device of claim 3, wherein the fluid delivery system further comprises a variable adjustable valve positioned between the outlet of the fluid container and the second one way valve, the variable adjustable valve being configured to produce the relatively constant volumetric flow rate.

7. A delivery device for accessing an IO space of a subject and delivering fluid to the IO space of the subject at a relatively constant volumetric flow rate over an extended period of time comprising:
(i) an outer cartridge comprising a coupling end and configured to hold an aspiration container;
(ii) an inner cartridge configured to be movably disposed in a proximal direction within the outer cartridge and to hold a fluid container containing the fluid, the inner cartridge comprising an aspiration plunger movably coupled to its distal end sized and configured to be movably disposed within the aspiration container;
(iii) a first actuator operable to activate the delivery device;
(iv) a plunger assembly sized and configured to be movably disposed within the fluid container from a first position to a second position;
(v) an energy storage member operable to produce a first force to drive the plunger assembly in a distal direction from the first position to the second position after activation of the delivery device;
(vi) a releasable retainer member configured to secure the plunger assembly in the first position and to release the plunger assembly after activation of the delivery device;
(vii) an IO access device disposed in the outer cartridge and configured to access the IO space of the subject, the IO access device comprising a second actuator operable to activate the IO access device, a tissue penetrator assembly releasably coupled to the coupling end of the outer cartridge and a driver operable to produce a second force to drive the tissue penetrator assembly into the IO space after actuation of the second actuator; and
(viii) a fluid delivery system having a first conduit configured to be fluidly coupled to an outlet of the aspiration container and a second conduit configured to be fluidly coupled to the outlet of the fluid container and a third conduit having an inlet in fluid communication with the first conduit and the second conduit and an outlet coupled to the tissue penetrator assembly.

8. The delivery device of claim 7, wherein the first actuator is a slide button.

9. The delivery device of claim 8, wherein the energy storage member is a spring.

10. The delivery device of claim 9, wherein the second actuator is a push button.

11. The delivery device of claim 10, wherein the driver is a spring.

12. A system comprising:
(A) a delivery device comprising:
(i) an outer cartridge comprising an aspiration container;
(ii) an inner cartridge configured to be movably disposed in a proximal direction within the outer cartridge comprising a fluid container containing a medicament and an aspiration plunger movably coupled to its distal end sized and configured to be movably disposed within the aspiration container;
(iii) a first actuator operable to activate the delivery device;
(iv) a plunger assembly sized and configured to be movably disposed within the fluid container from a first position to a second position;
(v) an energy storage member operable to produce a first force to drive the plunger assembly in a distal direction from the first position to the second position after activation of the delivery device;
(vi) a releasable retainer member coupled to the plunger assembly and configured to release the plunger assembly after activation of the delivery device; and
(vii) a catch mechanism; and
(B) an IO access device configured to be securely attached to the delivery device comprising:
(i) a housing comprising an inner coupling end;
(ii) a second actuator operable to activate the IO access device;
(iii) a tissue penetrator assembly disposed within the housing coupled to the coupling end of the housing and configured to be released from the coupling end after activation of the IO access device;
(iv) a driver operable to produce a second force to drive the tissue penetrator assembly into the IO space after activation of the IO access device;
(v) a fluid delivery system coupled to the tissue penetrator assembly and configured to be fluidly coupled to the outlet of the aspiration container and to the outlet of the fluid container; and
(vi) an engaging lock configured to be coupled to the catch mechanism of the delivery device to secure the IO access device to the delivery device.

13. A kit comprising:
(i) the delivery device of claim 1, and
(ii) a fluid container containing a fluid.

14. The kit of claim 13, wherein the fluid is a medicament.

15. The kit of claim 14, wherein the fluid comprises an analgesic, anti-inflammatory agent, anthelmintic, anti-arrhythmic agent, antibiotic, anticoagulant, antidepressant, antidiabetic agent, antiepileptic, antihistamine, antihypertensive agent, antimuscarinic agent, antimycobactefial agent, antineoplastic agent, antifibrinolytic, immunosuppressant, antithyroid agent, antiviral agent, anxiolytic sedative, astringent, beta-adrenoceptor blocking agent, blood product, cardiac inotropic agent, corticosteroid, cough suppressant, diagnostic agent, diuretic, dopaminergic, haemostatic, immunological agent, lipid regulating agent, muscle relaxant, parasympathomimetic, parathyroid calcitonin and biphosphonate, prostaglandin, radiopharmaceutical, sex hormone, anti-allergic agent, stimulant and anorexic, sympathomimetic, thrombolytic, thyroid agent, PDE IV inhibitor, NK3 inhibitor, ppar agent, NK-2 inhibitor, CSBP/RK/p38 inhibitor, antipsychotic, vasodilator or xanthine.

16. The kit of claim 14 wherein the medicament comprises tranexamic acid.

17. The kit of claim 13 further comprising an IO access device.

18. A system adapted to deliver a fluid into an IO space of a subject at a relatively constant volumetric flow rate over an extended period of time, the system comprising:
(A) a delivery device comprising:
(i) a fluid container having an open proximal end and a closed distal end with an orifice, the fluid container further containing the fluid;
(ii) a flow regulator comprising an inlet in fluid communication with the fluid container and an outlet;
(iii) a fluid channel in fluid communication with the outlet of the flow regulator,
(iv) a plunger assembly positioned at or near the proximal end of the fluid container and sized and configured to be movably disposed within the fluid container from a first position to a second position; and
(v) an energy storage member operable to produce a force on the plunger assembly to move the plunger assembly from the first position to the second position once activated;
(b) an IO access device comprising:
(i) a removable actuator,
(ii) a driver configured to be movably disposed from a first position to a second position when the actuator is activated,
(iii) a tissue penetrator assembly coupled to the driver and configured to access the IO space when the driver moves from the first position to the second position,
(iv) a fluid channel assembly coupled to the tissue penetrator assembly and configured to place the fluid channel assembly in fluid communication with the fluid channel when the driver moves from the first position to the second position, and
(c) an outer cartridge housing the fluid delivery system and substantially all of the IO access device
wherein in operation the IO access device is activated by activation of the actuator which moves the driver from the first position to second position allowing the tissue penetrator assembly to access the IO space and place the fluid channel assembly in fluid communication with the fluid channel and wherein the energy member is activated once the fluid channel assembly is in fluid communication with the fluid channel to provide the force to move the plunger assembly from the first position to the second position within the fluid container and dispense the fluid through the fluid channel to the fluid channel assembly and to the IO space at the relatively constant volumetric flow rate.

19. The fluid delivery system of claim 18, wherein the IO access device further comprises an energy storage member configured to provide a force on the actuator to remove the actuator after the actuator has been activated.

20. The fluid delivery system of claim 18, wherein the flow regulator comprises a helical channel configured to provide sufficient head loss which is equal to the force produced by the energy storage member.
